(19) United States
(12) Patent Application Publication
(10) Pub. No.: US 2011/0269758 A1
(43) Pub. Date: Nov. 3, 2011

(54) NAPHTHYRIDINONES AS PROTEIN KINASE INHIBITORS

(75) Inventors: Yufang Xiao, Lexington, MA (US); Bayard R. Huck, Sudbury, MA (US); Amanda E. Sutton, Hingham, MA (US); Xiaoling Chen, Chestnut Hill, MA (US); Srinivasa R. Karra, Pembroke, MA (US); Andreas Goutopoulos, Boston, MA (US)

(73) Assignee: MERCK PATENT GMBH, Darmstadt (DE)

(21) Appl. No.: 12/997,312

(22) PCT Filed: Jun. 29, 2009
(86) PCT No.: PCT/US2009/049035

§ 371 Date: Feb. 7, 2011

Related U.S. Application Data
(60) Provisional application No. 61/134,018, filed on Jul. 3, 2008.

(51) Int. Cl. A61K 31/5377; A61K 31/4375; A61K 31/519; C07D 413/14; A61P 37/06; A61P 17/06; A61P 9/10; A61P 31/04; A61P 31/12; C07D 471/04; A61P 35/00

(52) U.S. Cl. 514/234.5; 546/122; 514/300; 544/279; 514/264.11; 544/127

(57) ABSTRACT

Naphthyridinone derivative compounds that inhibit Aurora kinase enzymes are disclosed along with pharmaceutical compositions comprising these compounds and methods for synthesizing the same. Such compounds have utility in the treatment of proliferative diseases resulting from unregulated and/or disturbed Aurora kinases such as cancers, psoriasis, viral and bacterial infections, inflammatory and autoimmune diseases.

### FIELD OF THE INVENTION

The present invention relates to naphthyridinone compounds and their use as pharmacologically active agents capable of inhibiting protein kinases and aurora kinases

in particular, thereby inhibiting abnormal cellular proliferation and growth.

### BACKGROUND OF THE INVENTION

Protein kinases represent a large family of proteins, which play a central role in the regulation of a wide variety of cellular processes, and so maintain control over cellular function. These kinases includes Akt, Axl, Aurora A, Aurora B, Aurora C, dyrk2, epha2, fgfr3, flt-3, vegfr3, igf1r, IKK2, JNK3, Vegfr2, MEK1, MET, P70s6K, Plk1, RSK1, Src, TrkA, Zap70, cKit, bRaf, EGFR, Jak2, PI3K, NPM-Alk, c-Abl, BTK, FAK, PDGFR, TAK1, LimK, Flt3, Flt1, PDK1 and Erk, among others. Inhibition of such kinases has become an important therapeutic targeting tool.

Many diseases are associated with abnormal cellular responses triggered by protein kinase-mediated events. These diseases include cancers such as acute and chronic myelogenous leukemia (AML and CML), autoimmune, inflammatory, cardiovascular, neurological, myeloproliferative and neurodegenerative diseases, allergies and asthma, Alzheimer's disease and hormone-related diseases. Accordingly, there has been a substantial effort in medicinal chemistry to find protein kinase inhibitors that are effective as therapeutic agents.

The compounds of the present invention are novel, selective, and highly potent competitive inhibitors of Aurora kinases (A, B and C). The Aurora family of conserved serine/threonine kinases perform essential functions during cell division. The three mammalian paralogues are very similar in sequence, but differ significantly in their localization, function, substrates and regulatory partners.

Aurora A is mainly associated with the spindle poles during mitosis, where it is required for centrosome separation and maturation (Sausville E A. Nat. Med., (2004) 10:234-235). Spindle assembly requires that the targeting protein for XKLP 2, TPX2, targets Aurora A to spindle pole microtubules through a mechanism that requires Ran-GTP (Marumoto et al., Nature, (2005) 5:42-50). Aurora A also functions in meiosis by promoting oocyte maturation, polar-body extrusion, spindle positioning and exit from metaphase I. Regulation of Aurora A occurs through phosphorylation/dephosphorylation and degradation. Protein phosphatase 1 negatively regulates Aurora A and this interaction is modulated by TPX2.

Aurora B is a chromosomal-passenger protein with multiple functions in mitosis. Inner centromere protein (INCENP) and survivin, two other components of the passenger complex, function as targeting and regulatory factors for the kinase (Bishop J D and Shumacher J M. J. Biol. Chem. (2002) 277:27577-27580). Aurora B is required for phosphorylating histone H3, targeting condensing, and compacting normal chromosomes. It has also been recently shown to be essential for chromosome biorientation, kinetochore-microtubule interactions and the spindle-assembly checkpoint. Aurora B is essential for completion of cytokinesis.

Much less is known about Aurora C kinase, other than that it seems to be preferentially expressed in meiotic cells. During the cell cycle, Aurora kinases travel to their subcellular targets aided by their binding partner-substrates, INCENP, survivin and TPX2. This provides an additional level of regulation that might be essential for the choreography of mitotic events.

Aurora kinases are overexpressed in certain types of cancers, including colon, breast, and other solid-tumor cancers. The genes encoding the Aurora A and B kinases tend to be amplified in certain types of cancers, while the gene encoding the Aurora C kinase resides in a region of the chromosome that is subject to rearrangement and deletion. Aurora A has been associated with a variety of malignancies, including primary colon, colorectal, breast, stomach, ovarian, prostate, and cervical cancer, neuroblastoma, and other solid-tumor cancers (Warner et al. (2003) Molecular Cancer Therapeutics 2:589-95). Since Aurora A and B kinases are frequently elevated or overexpressed in human cancers makes them attractive targets for therapeutic intervention (Mountzios et al., Cancer Treatment Reviews (2008) 34:175-82; Gautschi et al., Clin. Cancer Res. (2008), 14(6):1639-48; Mortlock et al., Current Topics in Medicinal Chemistry (2005), 5:807-21).

Small molecule inhibitors of Aurora kinases have recently been reported, but their effect on cytokinesis has yet to be investigated in detail (Arora et al., J. Pharm. and Exptl. Therapeutics (2005), 315(3):971-79). For example, a high selective and potent small-molecule inhibitor of Aurora kinases, VX-680, blocks cell-cycle progression and induces apoptosis in a diverse range of human tumor types. This compound causes profound inhibition of tumor growth in a variety of in vivo xenograft models, leading to regression of leukemia, colon and pancreatic tumors at well-tolerated doses (Harrington et al., Nat. Med., (2004) 10: 262-267). Another novel cell cycle inhibitor, JNJ-7706621, shows potent inhibition of several cyclin-dependent kinases (CDKs) and Aurora kinases, and selectively blocked proliferation of tumor cells of various origins. At low concentrations, JNJ-7706621 slows the growth of cells and at high concentrations induces cytotoxicity. JNJ-7706621 treatment of cells has shown a delayed progression through G1 of the cell cycle and an arrest of the cell cycle at the G2-M phase (Emanuel et al., Cancer Res., (2005) 65:9038-9046). Additional cellular effects due to inhibition of Aurora kinases include endoreduplication and inhibition of histone H3 phosphorylation.

Naphthyridine compounds have been discovered that are useful in the treatment of rheumatic and respiratory diseases (WO 1993/13097, The Boots Company PLC; WO 2001/30779, Yamanouchi Pharmaceutical Co. Ltd.). Urea derivatives that have one or more nitrogen-containing aromatic rings, including a naphthyridinone ring, were found to be anti-angiogenic (U.S. Pat. No. 7,253,286 and WO 2002/032872, both to Eisai Co., Ltd.). American Cyanamid Company discovered certain cyano-substituted naphthyridine derivatives that are tyrosine kinase inhibitors, and thus are effective inhibitors of a variety of human tumor cell lines in vitro, such as the SKBR3 cell line (WO 2000/066583).

Accordingly, it is one object of the present invention to provide naphthyridine compounds that actively inhibit disturbed or unregulated Aurora kinase activity.

It is yet another object of the present invention to provide pharmaceutical compositions either individually or in kit form, and methods for using the same for treating proliferative disorders, such as cancers, psoriasis, viral and bacterial infections, vascular restinosis, inflammatory and autoimmune diseases, that result from unregulated and uncontrolled cellular proliferation.

It is still another object of the present invention to provide processes for preparing naphthyridine derivative compounds that actively inhibit unregulated Aurora kinase activity.

Additional objects, features and advantages of the present invention will become apparent to those skilled in the art from the following description and claims.

### SUMMARY OF THE INVENTION

The present invention relates to compounds that inhibit, regulate and/or modulate signal transduction of any the protein kinases, such as Akt, Axl, dyrk2, epha2, fgfr3, flt-3, vegfr3, igf1r, IKK2, JNK3, Vegfr2, MEK1, MET, P70s6K, Plk1, RSK1, Src, TrkA, Zap70, cKit, bRaf, EGFR, Jak2, PI3K, NPM-Alk, c-Abl, BTK, FAK, PDGFR, TAK1, LimK, Flt3, Flt1, PDK1 and Erk, and especially by the Aurora kinases A, B, and C. The invention also relates to compositions that comprise these compounds, and to methods for using the compounds in the treatment of Aurora kinase-related diseases and complaints. In a first aspect, the present invention provides a compound having a structure according to Formula I:

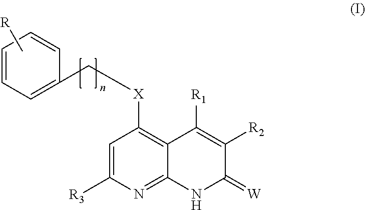

wherein:

X is NH, NH—C(═O), (—C═O)NH, NH—C(═O)NH, O, S, SO2NH, CH2, —C≡C—, or —CH═CH2;

W is O, S, CH2, or NH;

R is H, halo, cyano, nitro, alkyl, trifluoromethyl, heteroalkyl, OR′, SR′ and NR′ R″, where R′ and R″ each independently are H, alkyl, haloalkyl, alkylhalo, or heteroalkylr R is an heteroalkyl chain that optionally is bound at either end to adjoining carbon atoms of the phenyl ring to which it is attached, thereby forming a bicyclic ring structure;

R₁, R₂, R₃, each independently is H, SH or an ether or oxidated form of sulfur such as a sulfinyl, sulfonyl, sulfanyl, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid; wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group;

n is 1, 2, 3 or 4, with the proviso that n may=0 when X is other than oxygen; or

a pharmaceutically acceptable salt, prodrug, hydrate, solvate, tautomer, enantiomer or racemic mix thereof.

In a preferred embodiment, the compound according to Formula I is incorporated into a pharmaceutical formulation along with one or more pharmaceutically acceptable diluent, excipient, carrier, etc. Those of skill in the art will recognize the overlap in the terms “diluent”, “excipient” and “carrier”.

In a second aspect of the present invention, a compound of the general Formula II is provided,

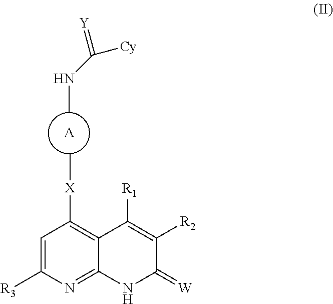

wherein:

X is NH, NH—CH2, NH—C(═O), (—C═O)NH, NH—C(═O)NH, O, S, SO₂NH, CH₂, —C≡C—, —CH═CH, or an heterocycle;

Y and W each independently is O, S, or NH;

A is a 3-7 membered ring, saturated or unsaturated, optionally having 1 or more heteroatoms and further optionally substituted;

Cy is selected from the group consisting of an unsubstituted or substituted cycloalkyl, bicycloalkyl such as norbornyl, aryl, heterocycle and heteroaryl;

R₁, R₂, _{R3} each independently is H, SH or an ether or oxidated form of sulfur such as a sulfide, sulfone, sulfane, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid; wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or

a pharmaceutically acceptable salt, prodrug, hydrate, solvate, tautomer, enantiomer or racemic mix thereof.

In a preferred embodiment, the compound according to Formula II is incorporated into a pharmaceutical formulation along with one or more pharmaceutically acceptable diluent, excipient, or carrier.

In a third aspect of the present invention, a compound of the general Formula III is provided,

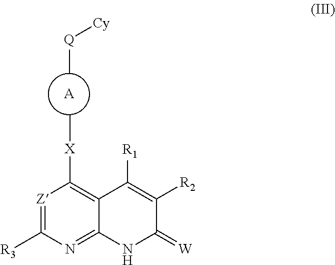

wherein:

X is NH, NH—C(═O), NH—CH2, (—C═O)NH, NH—C(═O)NH, O, S, SO₂NH, CH₂, —C≡C—, or —HC═CH—;

Q is NH(C═Y) or (C═Y)NH;

Y and W each independently is O, S, or NH;

Z′ is CH or N;

A is a 3-7 membered ring, saturated or unsaturated, optionally having 1 or more heteroatoms and further optionally substituted;

Cy is selected from the group consisting of an unsubstituted or substituted cycloalkyl, bicycloalkyl such as norbornyl, aryl, heterocycle and heteroaryl;

R₁, R₂, R₃, each independently is H, SH or an ether or oxidated form of sulfur such as a sulfide, sulfone, sulfane, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid, wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or

a pharmaceutically acceptable salt, prodrug, hydrate, solvate, tautomer, enantiomer or racemic mix thereof.

In a preferred embodiment, the compound according to Formula III is incorporated into a pharmaceutical formulation along with one or more pharmaceutically acceptable diluent, excipient, or carrier.

In a fourth aspect of the present invention, a compound of the general Formula IV is provided,

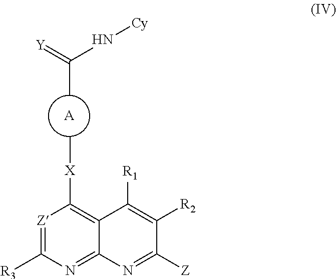

wherein:

X is NH, NH—C(═O), NHCH2, (—C═O)NH, NH—C(═O)NH, O, S, SO₂NH, CH₂, C≡C—, or —HC═CH—;

Y is O, S, or NH;

A is a 3-7 membered ring, saturated or unsaturated, optionally having 1 or more heteroatoms and further optionally substituted;

Cy is selected from the group consisting of an unsubstituted or substituted cycloalkyl, bicycloalkyl such as norbornyl, aryl, heterocycle and heteroaryl;

Z is H, SH, hydroxy, halo, amino, acyl, formyl, alkylamino-heterocycle, dialkylamino-heterocycle, alkylamino-alkylamino, dialkylamino-alkylamino, alkylamino-alkoxy, dialkylamino-alkoxy, heterocyclic alkoxy, C1-6 alkyl ester, phenyl, benzoyl, phenyl alkyl ketone, alkyl propanoyl, dialkyl alkanamide, acetic acid, or acetic acid amides;

Z′ is C or N;

------ denotes the presence or absence of a bond;

R1, R2, R3 each independently is H, SH or an ether or oxidated form of sulfur such as a sulfide, sulfone, sulfane, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid, wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or

a pharmaceutically acceptable salt, prodrug, hydrate, solvate, tautomer, enantiomer or racemic mix thereof.

In a preferred embodiment, the compound according to Formula IV is incorporated into a pharmaceutical formulation along with one or more pharmaceutically acceptable diluent, excipient, or carrier.

In a further aspect the invention provides a method for treating or preventing a disease or condition that is a member selected from cancers, tumor formation, angiogenesis, arteriosclerosis, ocular diseases, inflammatory diseases, arthritis, and restinosis, among others.

In a fifth aspect of the present invention, a compound intermediate of any of the Formulae V-VIII is provided,

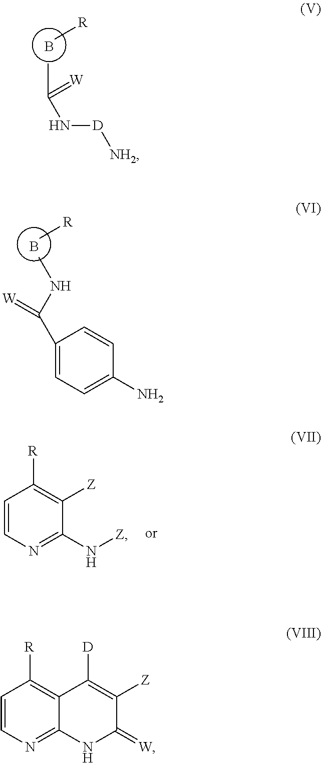

wherein:

B is 4-10 membered, saturated or unsaturated, ring that may be mono-, bi-, or tricyclic, and optionally may have one or more heteroatoms;

D is phenyl, a carbocycle, or a heterocycle, any of which optionally is substituted;

Z is H, SH and thioalkyl, hydroxy, halo, amino, acyl, formyl, alkylamino-heterocycle, dialkylamino-heterocycle, alkylamino-alkylamino, dialkylamino-alkylamino, alkylamino-alkoxy, dialkylamino-alkoxy, heterocyclic alkoxy, C1-6 alkyl ester, phenyl, benzoyl, phenyl alkyl ketone, alkyl propanoyl, dialkyl alkanamide, or acetic acid;

R is H, halo, cyano, nitro, alkyl, trifluoromethyl, an unsaturated or saturated ring having one or more heteroatoms such as piperazine or piperazine-C(═O)—, heteroalkyl, OR′, SR′ and NR′R″, where R′ and R″ each independently are H, alkyl, haloalkyl, alkylhalo, or heteroalkyl; or R is an heteroalkyl chain that optionally is bound at either end to adjoining carbon atoms of the phenyl ring to which it is attached, thereby forming a bicyclic ring structure; and

W is O, S, CH₂, or NH, or a tautomer or enantiomer thereof.

The compounds of Formulae V-VIII are useful in the syntheses of compounds of Formula I-IV.

Also included within the scope of the invention are compounds 1-86, and a pharmaceutically acceptable salt thereof.

Furthermore, the present invention provides pharmaceutical compositions and methods of modulating and/or inhibiting unregulated or disturbed Aurora kinase activity in order to treat or cure proliferative diseases comprising administering to a subject in need thereof an effective amount of a kinase inhibitor according to any of Formulae I, II, III, or IV. In particular, the compounds of the Formulae I, II, III and IV can be employed in the treatment of certain forms of cancer. The compounds of the Formulae I, II, III and IV furthermore can be used to provide additive or synergistic effects in certain existing cancer chemotherapies, and/or can be used to restore the efficacy of certain existing cancer chemotherapies and radiotherapies.

Additional embodiments of the present invention include: a compound according to any of Formulae I-IV for use as a medicament; use of the compound according to any of Formulae I-IV for the preparation of a medicament for the treatment of a subject in need of inhibiting a kinase protein; use of the compound according to any of Formulae I-IV for the preparation of a medicament for the suppression or reduction of cellular proliferation, including cancer metastasis, leukemias, and myeloproliferative diseases; a pharmaceutical composition comprising an effective amount of a compound of any of Formulae I, II, III or IV and a pharmaceutically acceptable carrier, excipient or diluent; a method of synthesizing the compounds of the present invention; a kit comprising a compound of Formula I, Formula II, Formula III or Formula IV, and a further pharmaceutically active ingredient; the combined use of a compound of Formula I, Formula II, Formula III, or Formula IV, together with further medicament active ingredient for the treatment of a subject in need of treatment for a kinase-related malfunction, and especially for diseases such as angiogenesis, cancer, and tumor formation.

### BRIEF DESCRIPTION OF THE DRAWINGS

Not applicable.

### DETAILED DESCRIPTION OF THE INVENTION

### I. Introduction

The present invention relates to compounds that inhibit, regulate and/or modulate signal transduction by any of the protein kinases and by the Aurora kinases in particular. The invention also relates to compositions that comprise these compounds, methods for using the compounds in the treatment of Aurora kinase-related diseases and complaints, and processes for synthesizing the compounds. In a first aspect, the present invention provides a compound having a structure according to Formula I:

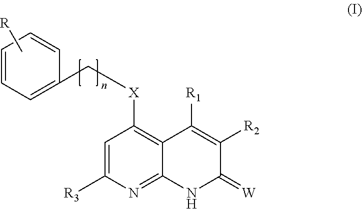

wherein:

X is NH, NH—C(═O)H, (—C═O)NH, NH—C(═O)NH, O, S, SO₂NH, CH₂, —C≡C—, or —HC═CH—;

W is O, S, or NH;

R is H, halo, cyano, nitro, alkyl, trifluoromethyl, heteroalkyl, OR′, SR′ and NR′R″, where R′ and R″ each independently are H, alkyl, haloalkyl, alkylhalo, or heteroalkyl; or R is an heteroalkyl chain that optionally is bound at either end to adjoining carbon atoms of the phenyl ring to which it is attached, thereby forming a bicyclic ring structure;

R₁, R₂, R₃ each independently is H, SH or an ether or oxidated form of sulfur such as a sulfide, sulfone, sulfane, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid, wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group;

n is 1, 2, 3 or 4, with the proviso that n may=0 when X is other than oxygen; or

a pharmaceutically acceptable salt, prodrug, hydrate, solvate, racemic mix, tautomer or enantiomer thereof.

In a first preferred embodiment of Formula I, R is F, X is NH, and W is O.

In a second preferred embodiment of Formula I, R is H, X is NH, and W is O.

In a third preferred embodiment of Formula I, R is CF₃, X is NH, and W is O.

In a fourth preferred embodiment, R is 3,4-fluoro-trifluoromethyl.

In a fifth preferred embodiment, R is Cl, X is NH, W is O, and n=0.

In a subembodiment of the fourth preferred embodiment of Formula I, R is 2,3-bis-CF₃.

In a second subembodiment of the fourth preferred embodiment, R is 2,4-bis-CF₃.

In a subembodiment of the fifth preferred embodiment, R simultaneously is both Cl and a 1,3-dioxoalkylene chain bound to the phenyl ring so as to form 1,3-dioxolane.

In yet another preferred embodiment the residues not designated in greater detail have the meaning indicated above, but in which
in Subformula Ia W is O, X is NH, n=0, and R is H;
in Subformula Ib W is O, X is NH, n=0, and R simultaneously is Cl and a 1,3-dioxoalkylene chain bound to the phenyl ring so as to form 1,3-dioxolane;
in Subformula Ic W is O, X is O, n=1, and R is H;
in Subformula Id W is S, X is CH₂, n=0, and R is Cl;
in Subformula Ie W is NH, X is CH₂, n=0, and R is F;
in Subformula If W is O, X is NH, n=1, and R is di-fluoro.

In a second aspect of the present invention, a compound of the general Formula II is provided,

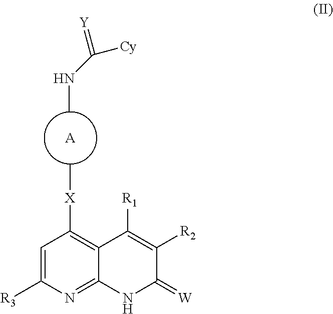

wherein:

X is NH, NH—C(═O)H, NH—CH₂, (—C═O)NH, NH—C(═O)NH, O, S, SO₂NH, CH₂, —C≡C—, —HC═CH—, or an heterocycle;

Y and W each independently is O, S, or NH;

A is a 3-7 membered ring, saturated or unsaturated, optionally having 1 or more heteroatoms and further optionally substituted;

Cy is selected from the group consisting of an unsubstituted or substituted cycloalkyl, bicycloalkyl such as norbornyl, aryl, heterocycle and heteroaryl; R₁, R₂, R₃ each independently is H, SH or an ether or oxidated form of sulfur such as a sulfide, sulfone, sulfane, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid; wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or

a pharmaceutically acceptable salt, prodrug, hydrate, solvate, tautomer, racemic mix, or enantiomer thereof.

In a first preferred embodiment of Formula II, X is NH, W is O, Y is O, A is phenyl, Cy is phenyl, and R1, R2, R3 and R, each independently, is H.

In a first subembodiment of the first preferred embodiment, Cy is 2-, 3- or 4-fluorophenyl.

In a second subembodiment of the first preferred embodiment, Cy is 2- or 4-trifluoromethylphenyl.

In a third subembodiment of the first preferred embodiment, Cy is 2,4-bis-(trifluoromethyl)phenyl.

In a fourth subembodiment of the first preferred embodiment, Cy is 2-fluoro-3-trifluoromethylphenyl or 2-fluoro-4-trifluoromethylphenyl.

In a fifth subembodiment of the first preferred embodiment, Cy is 2,4-, 2,6-, 3,4- or 3,5-difluorophenyl.

In a second preferred embodiment of Formula II, X is NH, W is O, Y is O, A is phenyl, Cy is cyclohexanyl, and R₁, R₂, R₃ and R, each independently, is H.

In a third preferred embodiment of Formula II, X is O, W is O, Y is O, A is phenyl, Cy is phenyl, and R₁, R₂, R₃ and R, each independently, is H.

In a fourth preferred embodiment of Formula II, X is NH, W is O, Y is O, A is phenyl, Cy is naphthyl, and R₁, R₂, R₃ and R, each independently, is H.

In a third aspect of the present invention, a compound of the general Formula III is provided,

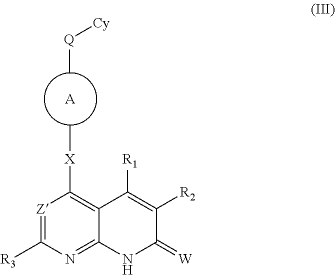

wherein:

X is NH, NH—C(═O)H, NH—CH₂, (—C═O)NH, NH—C(═O)NH, O, S, SO₂NH, CH₂, —C≡C—, or —HC═CH—;

Q is NH(C═Y) or (C═Y)NH;

Y and W each independently is O, S, or NH;

Z′ is CH or N;

A is a 3-7 membered ring, saturated or unsaturated, optionally having 1 or more heteroatoms and further optionally substituted;

Cy is selected from the group consisting of an unsubstituted or substituted cycloalkyl, bicycloalkyl such as norbornyl, aryl, heterocycle and heteroaryl;

R₁, R₂, R₃ each independently is H, SH or an ether or oxidated form of sulfur such as a sulfide, sulfone, sulfane, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid, wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or

a pharmaceutically acceptable salt, prodrug, hydrate, solvate, tautomer, racemic mix, or enantiomer thereof.

In a preferred embodiment of Formula III, X is NH, A and Cy each independently is phenyl, W and Y each independently is O, and R₁, R₂ and R₃ are H.

In a subembodiment of the preferred embodiment, Cy is methoxyphenyl.

In a second subembodiment of the preferred embodiment, Cy is methylphenyl.

In a third subembodiment of the preferred embodiment, Cy is 2-fluoro-4-trifluoromethylphenyl.

In a fourth subembodiement of the preferred embodiment, Cy is 4-chlorophenyl.

In a fifth subembodiment of the preferred embodiment, Cy is 4-trifluoromethoxyphenyl.

In a sixth subembodiment of the preferred embodiment, Cy is 2,4-, 2,6- or 3,4-dichlorophenyl.

In a second preferred embodiment of Formula III, X is NH, A is phenyl, Cy is naphthyl, W and Y each independently is O, and R₁, R₂ and R₃ are H.

In yet another preferred embodiment the residues not designated in greater detail have the meaning indicated above, but in which in Subformula IIIa Q is NH(C═O), W is O, X is NH, and A and Cy are phenyl;

in Subformula IIIb Q is NH(C═O) and Cy is methoxyphenyl;

in Subformula Inc Q is NH(C═O) and Cy is methylphenyl;

in Subformula IIId Q is NH(C═O) and Cy is fluoro, trifluoromethyl phenyl;

in Subformula IIIe Q is NH(C═O) and Cy is chlorophenyl or dichlorophenyl;

in Subformula IIIf Q is NH(C═O) and Cy is naphthyl;

in Subformula IIIg Q is NH(C═O) and Cy is norbornyl;

in Subformula IIIb Q is NH(C═O) and Cy is trifluoromethoxyphenyl;

in Subformula IIIj Q is (C═O)NH, W is O, X is NH, and A and Cy are phenyl;

in Subformula IIIk Q is (C═O)NH and Cy is methoxyphenyl;

in Subformula IIIm Q is (C═O)NH and Cy is methylphenyl;

in Subformula IIIn Q is (C═O)NH and Cy is fluoro, trifluoromethyl phenyl;

in Subformula IIIo Q is (C═O)NH and Cy is chlorophenyl or dichlorophenyl;

in Subformula IIIp Q is (C═O)NH and Cy is naphthyl;

in Subformula IIIq Q is (C═O)NH and Cy is norbornyl;

in Subformula IIIr Q is (C═O)NH and Cy is trifluoromethoxyphenyl.

In a fourth aspect of the present invention, a compound of the general Formula IV is provided,

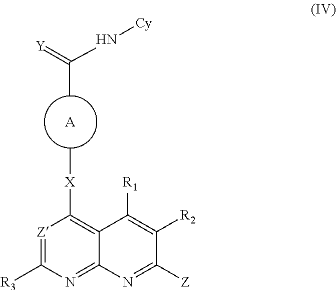

wherein:

X is NH, NH—C(═O), NH—CH₂, (—C═O)NH, NH—C(═O)NH, O, S, SO₂NH, CH₂, C≡C—, or —HC═CH—;

Y is O, S, or NH;

A is a 3-7 membered ring, saturated or unsaturated, optionally having 1 or more heteroatoms and further optionally substituted;

Cy is selected from the group consisting of an unsubstituted or substituted cycloalkyl, bicycloalkyl such as norbornyl, aryl, heterocycle and heteroaryl;

Z is H, SH, hydroxy, halo, amino, acyl, formyl, alkylamino-heterocycle, dialkylamino-heterocycle, alkylamino-alkylamino, dialkylamino-alkylamino, alkylamino-alkoxy, dialkylamino-alkoxy, heterocyclic alkoxy, C₁₋₆ alkyl ester, phenyl, benzoyl, phenyl alkyl ketone, alkyl propanoyl, dialkyl alkanamide, acetic acid, or acetic acid amides;

Z′ is C or N;

------ denotes the presence or absence of a bond;

R₁, R₂, R₃ each independently is H, SH or an ether or oxidated form of sulfur such as a sulfide, sulfone, sulfane, sulfinimide, or sulfonamide; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid; wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or

a pharmaceutically acceptable salt, prodrug, hydrate, solvate, tautomer, racemic mix, or enantiomer thereof.

In a first preferred embodiment of Formula IV, X is NH, A is phenyl, Y is O, Cy is phenyl, R₁, R₂ and R₃ each independently is H, and Z is chloro.

In a second preferred embodiment of Formula IV, X is NH, A and Cy each independently is phenyl, Y is O, R₁, R₂ and R₃ each independently is H, and Z is dimethylamino-piperidine.

In a third preferred embodiment of Formula IV, X is NH, A and Cy each independently is phenyl, Y is O, R₁, R₂ and R₃ each independently is H, and Z is dimethylamino-ethylamine.

In a subembodiment of the third preferred embodiment, Z is dimethylamino-propylamine.

In a fourth preferred embodiment of Formula IV, X is NH, A and Cy each independently is phenyl, Y is O, R₁, R₂ and R₃ each independently is H, and Z is dimethylamino-pyrrolidine.

In a fifth preferred embodiment of Formula IV, X is NH, A and Cy each independently is phenyl, Y is O, R₁, R₂ and R₃ each independently is H, and Z is dimethylamino-ethoxy.

In a subembodiment of the fifth preferred embodiment, Z is dimethylamino-propoxy.

In a sixth preferred embodiment of Formula IV, X is NH, A and Cy each independently is phenyl, Y is O, R₁, R₂ and R₃ each independently is H, and Z is pyrrolidinyl-ethoxy.

In a seventh preferred embodiment of Formula IV, X is NH, A and Cy each independently is phenyl, Y is O, R₁, R₂ and R₃ each independently is H, and Z is morpholinyl-propoxy.

In a subembodiment of the seventh preferred embodiment, Z is morpholinyl-ethoxy.

In yet another preferred embodiment the residues not designated in greater detail have the meaning indicated above, but in which in Subformula IVa R1 is H, X is NH, A is phenyl, Cy is phenyl, and Z is 4-dimethyl amino-piperidine;
in Subformula IVb R₁ is H, X is NH, A is phenyl, Cy is phenyl, and Z is dimethylamino-ethylamine;
in Subformula IVc Z is dimethylamino-propylamine;
in Subformula IVd Z is dimethylamino-pyrrolidine;
in Subformula IVe Z is dimethylaminoethoxy or dimethylaminopropoxy;
in Subformula IVf Z is pyrrolidinyl ethoxy or pyrrolidinyl propoxy;
in Subformula IVg W is O, Y is O, X is NH, A is phenyl, Cy is difluorophenyl, R₁ is phenyl and/or carboxylic acid;
in Subformula IVh W is O, Y is O, X is NH, A is phenyl, Cy is difluorophenyl, and R₁ is dimethylamino ethyl carboxylic acid amide;
in Subformula IVj W is O, Y is O, X is NH, A is phenyl, Cy is difluorophenyl, and Z is dihydroxypropyl carboxylic acid amide;
in Subformula IVk W is O, Y is O, X is NH, A is phenyl, Cy is difluorophenyl, and R₂ is methyl acetic acid;
in Subformula IVm W is O, Y is O, X is NH, A is phenyl, Cy is difluorophenyl, and R₂ is hydroxyethyl acetic acid amide.

Also encompassed by the present invention are methods of treating a subject in need of inhibiting a kinase protein comprising administering to the subject an effective amount of a kinase inhibitor according to Formulae I, II, III or IV, or any mixture of thereof.

In a preferred embodiment, the compound according to Formula I, Formula II, Formula III or Formula IV is incorporated into a pharmaceutical formulation along with one or more pharmaceutically acceptable diluent, excipient, or carrier, and which further optionally may be packaged as a kit.

In a further aspect the invention provides a method for treating or preventing a disease or condition that is a member selected from cancers such as acute or chronic myelogenous leukemia, tumor formation, tumor angiogenesis, myeloproliferative disease, arteriosclerosis, ocular diseases, inflammatory diseases, arthritis, and restinosis, among others. The method includes administering to a subject in need thereof a therapeutically effective amount of a compound of Formula I, Formula II, Formula III, or Formula IV, or a pharmaceutically acceptable salt, prodrug, enantiomer, tautomer, hydrate, solvate or racemic mixture thereof.

In a fifth aspect of the present invention, a compound intermediate of the Formulae V-VIII is provided,

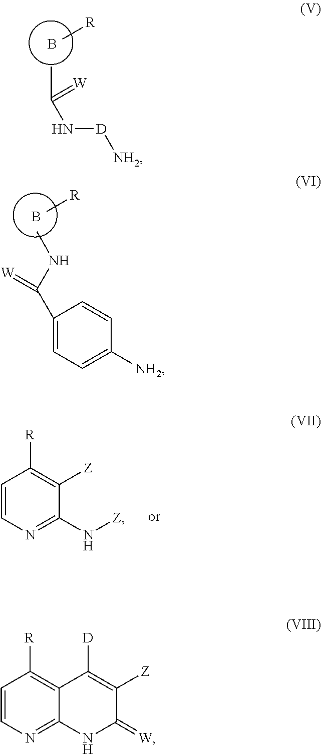

wherein:

B is 4-10 membered, saturated or unsaturated, ring that may be mono-, bi-, or tricyclic, and optionally may have one or more heteroatoms;

D is phenyl, a carbocycle, or a heterocycle, any of which optionally is substituted;

Z is H, SH, hydroxy, halo, amino, acyl, formyl, alkylamino-heterocycle, dialkylamino-heterocycle, alkylamino-alkylamino, dialkylamino-alkylamino, alkylamino-alkoxy, dialkylamino-alkoxy, heterocyclic alkoxy, C₁₋₆ alkyl ester, phenyl, benzoyl, phenyl alkyl ketone, alkyl propanoyl, dialkyl alkanamide, or acetic acid;

R is H, halo, cyano, nitro, alkyl, trifluoromethyl, an unsaturated or saturated ring having one or more heteroatoms such as piperazine or piperazine-C(═O)—, heteroalkyl, OR′, SR′ and NR′R″, where R′ and R″ each independently are H, alkyl, haloalkyl, alkylhalo, or heteroalkyl; or R is an heteroalkyl chain that optionally is bound at either end to adjoining carbon atoms of the phenyl ring to which it is attached, thereby forming a bicyclic ring structure; and

W is O, S, CH₂, or NH, or a tautomer or enantiomer thereof.

In a preferred embodiment, B is a phenyl, naphthyl or cyclohexyl moiety; R is one or more hydrogen, halo or trifluoromethyl groups; W is oxygen; D is phenyl or a heterocycle, preferably pyrimidine; and Z is hydrogen, formyl, t-butyl hydroxypropanoate, benzoyl, acetic acid or dimethyl propanamide. The compounds of Formulae V-VIII are useful in the syntheses of compounds of Formulae I-IV.

Also included within the scope of the invention are compounds 1-80, and a pharmaceutically acceptable salt thereof.

Additional embodiments of the present invention include: a compound according to any of Formulae I, II, III, or IV for use as a medicament; use of the compound according to any of Formulae I, II, III, or IV for the preparation of a medicament for the treatment of a subject in need of inhibiting a kinase protein; and use of the compound according to any of Formulae I, II, III, or IV for the preparation of a medicament for the suppression or reduction of cellular proliferation in single-site or metastatic cancers.

The present invention also encompasses a compound according to any of Formulae I, II, III, or IV, or pharmaceutically acceptable derivatives, solvates, salts, tautomers and stereoisomers thereof, including mixtures thereof in all ratios, for use in therapy, such as treating a subject in need of inhibiting a kinase protein, wherein the subject has a proliferative or an inflammatory disease.

A method of synthesizing the compounds of the present invention also is encompassed within the present invention.

The present invention also is related to the combined use of a compound of any of Formulae I, II, III, or IV together with further medicament active ingredient for the treatment of a subject in need of treatment for a kinase-related malfunction, and especially for diseases such as angiogenesis, cancers such as acute or chronic myelogenous leukemia, myeloproliferative disease, tumor formation, growth and propagation, arteriosclerosis, ocular diseases, such as age-induced macular degeneration, choroidal neovascularisation and diabetic retinopathy, inflammatory diseases, arthritis, thrombosis, fibrosis, glomerulonephritis, neurodegeneration, psoriasis, restenosis, wound healing, transplant rejection, metabolic diseases, autoimmune diseases, cirrhosis, diabetes and vascular and immune diseases in mammals.

The compounds of the present invention especially are useful as Aurora kinase inhibitors for the treatment of solid tumors characterized by having Aurora kinases that are strongly expressed or overexpressed. Such solid tumors include, among others, monocytic leukaemia, brain, breast, pancreatic, ovarian, urogenital, lymphatic system, stomach, laryngeal and lung carcinoma, including lung adenocarcinoma and small-cell lung carcinoma.

Furthermore, the present invention provides pharmaceutical compositions and methods of modulating and/or inhibiting unregulated or disturbed Aurora kinase activity in order to treat or cure proliferative diseases including all types of cancers comprising administering to a subject in need thereof an effective amount of a kinase inhibitor according to any of Formulae I, II, III, or IV. In particular, the compounds of the Formulae I, II, III, or IV are useful in the treatment of certain forms of cancer. The compounds of the Formulae I, II, III, or IV furthermore can be used to provide additive or synergistic effects in certain existing cancer chemotherapies, and/or can be used to restore the efficacy of certain existing cancer chemotherapies and radiotherapies.

The compounds of the present invention especially are useful as Aurora kinase inhibitors for the treatment of solid tumors characterized by having Aurora kinases that are strongly expressed or overexpressed. Such solid tumours include, among others, monocytic leukaemia, brain, breast, pancreatic, ovarian, urogenital, lymphatic system, stomach, laryngeal and lung carcinoma, including lung adenocarcinoma and small-cell lung carcinoma.

The compounds of the present invention have an antiproliferative action in vivo in a xenotransplant tumor model by their inhibitory action on cell division. Thus, when they are administered to a patient having a hyperproliferative disease, such as a myeloproliferative disease, these compounds inhibit tumor growth, reduce inflammation associated with a lymphoproliferative disease, inhibit transplant rejection, inhibit neurological damage due to tissue repair, etc. The present compounds are suitable for prophylactic or therapeutic purposes. The prevention of proliferation is achieved by administration of the compounds according to the invention prior to the development of overt disease, for example to prevent the growth of tumors, prevent metastatic growth, diminish restenosis associated with cardiovascular surgery, etc. Alternatively, the compounds are used for the treatment of ongoing diseases by stabilizing or improving the clinical symptoms of the patient.

### II. Definitions

As used herein, a description of the compounds of the invention in every case includes a pharmaceutically acceptable salt, solvate, hydrate, prodrug, tautomer, enantiomer, stereoisomer, analog or derivative thereof, including mixtures thereof in any ratios.

Where substituent groups are specified by their conventional chemical formulae, written from left to right, they optionally encompass substituents resulting from writing the structure from right to left, e.g., —CH₂O— optionally also recites —OCH₂—.

The term “alkyl”, by itself or as part of another substituent, unless otherwise stated means a saturated or unsaturated, unbranched (linear) or branched chain, or a cyclic hydrocarbon radical, or combination thereof, having 1, 2, 3, 4, 5, 6, 7, 8, 9 or 10 C atoms. The term preferably denotes methyl, ethyl, propyl, isopropyl, butyl, isobutyl, sec-butyl or tert-butyl, pentyl, or hexyl, and includes cycloalkyl and bicycloalkyl, e.g. cyclopropyl, cyclobutyl, cyclopentyl, cyclohexyl, cycloheptyl, norbornane, and the like. An unsaturated hydrocarbon radical is one having one or more double bonds or triple bonds. Examples of unsaturated alkyl groups include vinyl, 2-propenyl, crotyl, 2-isopentenyl, 2-butadienyl, 2,4-pentadienyl, ethynyl, 1-propenyl, 3-propenyl, 3-butynyl, and isomers and homologs thereof. One to seven hydrogen atoms in an alkyl chain as defined may be replaced by F, Cl and/or Br, and/or one or two CH2 groups may be replaced by O, S, SO, SO₂ and/or CH═CH groups.

The term “alkylene” denotes an optionally substituted, unbranched (linear) or branched chain that by itself or as part of another substituent means a divalent radical derived from an alkane, as exemplified by —CH₂CH₂CH₂—. “Alkylene” preferably denotes methylene, ethylene, propylene, isopropylene, butylene, isobutylene, sec-butylene or tert-butylene, pentylene, 1-, 2- or 3-methylbutylene, 1,1-, 1,2- or 2,2-dimethylpropylene, 1-ethylpropylene, hexylene, 1-, 2-, 3- or 4-methylpentylene, 1,1-, 1,2-, 1,3-, 2,2-, 2,3- or 3,3-dimethylbutylene, 1- or 2-ethylbutylene, 1-ethyl-1-methylpropylene, 1-ethyl-2-methylpropylene, 1,1,2- or 1,2,2-trimethylpropylene, or difluoromethylene. Especially preferred is an alkylene having 1, 2, 3, 4, 5 or 6 C atoms, preferably methylene, ethylene, propylene, isopropylene, butylene, isobutylene, sec-butylene, tert-butylene, pentylene, hexylene, difluoromethylene, tetrafluoroethylene or 1,1-difluoroethylene.

A “cyclic alkylene” (“cycloalkylene”) preferably denotes cyclopropylene, cyclobutylene, cyclopentylene, cyclohexylene or cycloheptylene.

The term “aryl” means, unless otherwise stated, means a polyunsaturated, aromatic, single ring or multiple rings, preferably from 1 to 3 rings, the latter of which are fused together or linked covalently. The term “aryl” denotes, for example, phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)phenyl, o-, m- or p-(N-ethylamino)phenyl, o-, m- or p-(N,N-diethylamino)phenyl, o-, m- or p-fluorophenyl including difluorophenyl, o-, m- or p-bromophenyl including dibromophenyl, o-, m- or p-chlorophenyl including dichlorophenyl, o-, m- or p-(methylsulfonamido)phenyl, o-, m- or p-(methylsulfonyl)phenyl, o-, m- or p-methylsulfanylphenyl, o-, m- or p-cyanophenyl, o-, m- or p-carboxyphenyl, o-, m- or p-methoxycarbonylphenyl, o-, m- or p-formylphenyl, o-, m- or p-acetylphenyl, o-, m- or p-aminosulfonylphenyl, o-, m- or p-(morpholin-4-ylcarbonyl)phenyl, o-, m- or p-(morpholin-4-ylcarbonyl)phenyl, o-, m- or p-(3-oxomorpholin-4-yl)phenyl, o-, m- or p-(piperidinylcarbonyl)phenyl, o-, m- or p-[2-(morpholin-4-yl)ethoxy]phenyl, o-, m- or p[3-(N,N-diethylamino)propoxy]phenyl, o-, m- or p-[3-(3-diethylamino-propyl)ureido]phenyl, o-, m- or p-(3-diethylaminopropoxycarbonylamino)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethyl-aminophenyl, 2,3-diaminophenyl, 2,3,4-, 2,3,5-, 2,3,6-, 2,4,6- or 3,4,5-tri-chlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl.

In a preferred embodiment, “aryl” preferably denotes a phenyl that is unsubstituted or mono-, di- or trisubstituted independently by one or more halogens, OR, CN or (C═O)NH₂ where R is H or alkyl.

The term “heteroaryl” refers to an aryl ring that contains from one to four heteroatoms selected from N, O, S, Si, P and B, wherein the nitrogen and sulfur atoms are optionally oxidized, and the nitrogen atom(s) are optionally quaternized. A heteroaryl group can be attached to the remainder of the molecule through a carbon or heteroatom.

Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 7-azaindole, 1-isoquinolyl, 5-isoquinolyl, 2-quinoxalinyl, 5-quinoxalinyl, 3-quinolyl, 6-quinolyl, 1-piperidinyl, 3-benzofuranyl, and 4-benzodioxinyl. Substituents for each of the above noted aryl and heteroaryl ring systems are selected from the group of acceptable substituents described below.

For brevity, the term “aryl” when used in combination with other terms, such as for example, aryloxy, arylthioxy, or arylalkyl, optionally includes both aryl and heteroaryl rings as defined above. Thus, the term “arylalkyl” optionally includes those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). Each of the terms “alkyl,” “heteroalkyl,” “aryl” and “heteroaryl” optionally include unsubstituted, mono-, di- or tri-unsubstituted forms of the indicated radical.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

Substituents for the alkyl and heteroalkyl radicals, including those groups often referred to as alkenyl, heteroalkylene, heteroalkenyl, alkynyl, cycloalkyl, heterocycloalkyl, cycloalkenyl, and heterocycloalkenyl, are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, and —R₁, wherein R₁ is —OH, O-alkyl, —CN, -halo, —C(O)OH, —C(O)O(alkyl), —C(O)NH₂, —C(O)NH(alkyl), —C(O)N(alkyl)₂, —CH₂OH, —CH₂O(alkyl), —CH₂NH₂, —CH₂NH(alkyl), —CH₂N(alkyl)₂, —SO₂OH, —SO₂O(alkyl), —SO₂NH₂, —SO₂NH(alkyl), and —SO₂N(alkyl)₂. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted aryl, substituted or unsubstituted heteroaryl, substituted or unsubstituted heterocycloalkyl, —OH, —O-alkyl, —CN, -halo, —C(O)OH, —C(O)O(alkyl), —C(O)NH₂,

—C(O)NH(alkyl), —C(O)N(alkyl)₂, —CH₂OH, —CH₂O(alkyl), —CH₂NH₂, —CH₂NH(alkyl), —CH₂N(alkyl)₂, —SO₂OH, —SO₂O(alkyl), —SO₂NH₂, —SO₂NH(alkyl), and —SO₂N(alkyl)₂.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non aromatic rings. Examples of “fused ring systems” are naphthalenes, indoles, quinolines, chromenes, substituted and unsubstituted norbornanes and norbornenes, and the like.

The term “treatment” as used herein refers both to prevention of a particular disease or treatment of a pre-existing condition.

The phrase “therapeutically effective amount” as used herein means that amount of a compound, material, or composition comprising a compound of the present invention which is effective for producing some desired therapeutic effect by simultaneous blocking or inhibiting of Aurora kinase receptors in a mammal, thereby blocking the biological consequences of that pathway in the treated cells, at a reasonable benefit/risk ratio applicable to any medical treatment.

The term “pharmaceutically acceptable salts” includes salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., J. Pharma. Science 1977, 66: 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. For instance, prodrugs for carboxylic acid analogs of the invention include a variety of esters. In an exemplary embodiment, the pharmaceutical compositions of the invention include a carboxylic acid ester. In another exemplary embodiment, the prodrug is suitable for treatment/prevention of those diseases and conditions that require the drug molecule to cross the blood brain barrier. In a preferred embodiment, the prodrug enters the brain, where it is converted into the active form of the drug molecule. Additionally, prodrugs can be converted to the compounds of the present invention by chemical or biochemical methods in an ex vivo environment.

For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are of use in the methods contemplated by the present invention and are intended to be within the scope of the present invention. “Compound or a pharmaceutically acceptable salt, hydrate, polymorph or solvate of a compound” intends the inclusive meaning of “or”, in that materials meeting more than one of the stated criteria are included, e.g., a material that is both a salt and a solvate is encompassed.

As used herein, the term “heteroatom” includes oxygen (O), nitrogen (N), sulfur (S), silicon (Si), boron (B), and phosphorus (P).

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to,

—CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. A “cycloalkyl” or “heterocycloalkyl” substituent may be attached to the remainder of the molecule directly or through a linker, wherein the linker is preferably alkyl. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, difluoromethyl, fluoromethyl, 2,2,2-trifluoroethyl, 4-chlorobutyl, 3-bromopropyl, and the like.

The terms “TEA”, “DMF”, “LDA”, “DCM” and “TFA” used herein as reagents in the syntheses of compounds of the invention mean “tetraethylammonia”, “N,N-dimethylformamide”, “lithium diisopropylamine”, “dichloromethane” and “trifluoroacetic acid”, respectively.

Certain compounds of the present invention possess asymmetric carbon atoms (optical centers) or double bonds; the racemates, diastereomers, geometric isomers and individual isomers are encompassed within the scope of the present invention. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

Likewise, all tautomeric forms are included.

The term “host” or “patient in need thereof” as used herein may be any mammalian species, for example a primate species, particularly humans; rodents; rabbits; horses, cows, sheep, dogs, cats, etc. Animal models are of interest for veterinary treatment and for experimental investigations, providing a model for treatment of human disease.

The susceptibility of a particular cell to treatment with the compounds according to the invention was determined by in vitro tests. Typically, a culture of the cell was combined with a compound according to the invention at various concentrations for a period of time that was sufficient to allow the active agents to induce cell death or to inhibit migration, usually between about one hour and one week. In vitro testing was carried out using cultivated cells from a biopsy sample. The viable cells remaining after the treatment then were counted.

Drug dosage depends upon the specific compound used, the specific disease, the patient status, etc. A therapeutic dose is typically sufficient considerably to reduce the undesired cell population in the target tissue while the viability of the patient is maintained. The treatment is generally continued until a reduction in cell population has occurred, for example, at least about 50% reduction in the cell burden, and may be continued until essentially no more undesired cells are detected in the body.

### Pharmaceutical Compositions

While compounds of the present invention can be administered as the raw chemical, it is preferable to present them as a pharmaceutical composition. According to a further aspect, the present invention provides a pharmaceutical composition comprising a compound of Formula I, II, III or IV, or a pharmaceutically acceptable salt, hydrate or solvate thereof, together with one or more pharmaceutical carrier and optionally one or more other therapeutic ingredients. The carrier(s) are “acceptable” in the sense of being compatible with the other ingredients of the formulation and not deleterious to the recipient thereof. The term “pharmaceutically acceptable carrier” includes vehicles, diluents, excipients and other elements appropriate for incorporation into a pharmaceutical formulation.

A formulation of the compound or composition includes any suitable for parenteral (including subcutaneous, intradermal, intramuscular, intravenous, peritoneal and intraarticular), rectal, ionotophoretic, intranasal, inhalation, and oral (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound or a pharmaceutically acceptable salt or solvate thereof (“active ingredient”) with the carrier that constitutes one or more accessory ingredients. In general, the formulations are prepared by uniformly and intimately bringing into association the active ingredient with liquid carriers or finely divided solid carriers or both and then, if necessary, shaping the product into the desired formulation. Oral formulations are well known to those skilled in the art, and general methods for preparing them are found in any standard pharmacy school textbook, for example, Remington: The Science and Practice of Pharmacy., A. R. Gennaro, ed. (1995), the entire disclosure of which is incorporated herein by reference.

Pharmaceutical compositions containing compounds of Formulae I. II, III or IV may be conveniently presented in unit dosage form and prepared by any of the methods well known in the art of pharmacy. Preferred unit dosage formulations are those containing an effective dose, or an appropriate fraction thereof, of the active ingredient, or a pharmaceutically acceptable salt thereof. The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose ranges from about 0.1 mg per day to about 7000 mg per day, preferably about 1 mg per day to about 100 mg per day, and more preferably, about 25 mg per day to about 50 mg per day, in single or divided doses. In some embodiments, the total daily dose may range from about 50 mg to about 500 mg per day, and preferably, about 100 mg to about 500 mg per day. It is further recommended that children, patients over 65 years old, and those with impaired renal or hepatic function, initially receive low doses and that the dosage is titrated based on individual responses and/or blood levels. It may be necessary to use dosages outside these ranges in some cases, as will be apparent to those in the art. Further, it is noted that the clinician or treating physician knows how and when to interrupt, adjust or terminate therapy in conjunction with individual patient's response.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compressing or molding the compound of Formula I, II, III or IV, optionally using one or more additional ingredient. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein. Oral and parenteral sustained release drug delivery systems are well known to those skilled in the art, and general methods of achieving sustained release of orally or parenterally administered drugs are found, for example, in Remington, THE SCIENCE AND PRACTICE OF PHARMACY, 21^{ST} Ed., (1995) Pages 1660-75. It should be understood that in addition to the ingredients particularly mentioned above, the formulations of this invention may include other agents conventional in the art having regard to the type of formulation in question, for example those suitable for oral administration may include flavoring agents.

Formulations for parenteral administration include aqueous and non-aqueous sterile injection solutions that may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents, while formulations for oral administration also may include flavoring agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described. Formulations for rectal administration may be presented as a suppository with the usual carriers such as cocoa butter or polyethylene glycol. Formulations for topical administration in the mouth, for example, buccally or sublingually, include lozenges comprising the active ingredient in a flavored basis such as sucrose and acacia or tragacanth, and pastilles comprising the active ingredient in a basis such as gelatin and glycerin or sucrose and acacia.

The pharmaceutically acceptable carrier may take a wide variety of forms, depending on the route desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets, with the solid oral preparation being preferred over the liquid preparations. Preferred solid oral preparations are tablets or capsules, because of their ease of administration. If desired, tablets may be coated by standard aqueous or nonaqueous techniques. Oral and parenteral sustained release dosage forms may also be used.

Exemplary formulations, are well known to those skilled in the art, and general methods for preparing them are found in any standard pharmacy school textbook, for example, Remington, The Science and Practice of Pharmacy, 21st Ed., (1995) Lippincott.

One aspect of the present invention contemplates the treatment of the disease/condition with the pharmaceutically active agent that may be sold in kit form. The kit comprises a compound of the present invention contained within a syringe, box, bag, and the like. Typically, the kit comprises directions for the administration of the compound. The kit form is particularly advantageous when different dosage concentrations and/or forms (e.g., oral and parenteral) are sold, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). They generally consist of a sheet of relatively stiff material covered with a foil of a preferably transparent plastic material. During the packaging process recesses are formed in the plastic foil. The recesses have the size and shape of the tablets or capsules to be packed. The tablets or capsules are placed in the recesses and the sheet of relatively stiff material is sealed against the plastic foil at the face of the foil which is opposite from the direction in which the recesses were formed. Particular dosage information normally is stamped onto each blister pack.

In another specific embodiment of the invention, a dispenser designed to dispense the daily doses one at a time in the order of their intended use is provided.

### III. Methods of Treatment or Prevention

In a further aspect the invention provides a method for treating or preventing a disease or condition that is a member selected from kinase-related malfunction, and especially for diseases such as angiogenesis, cancers, tumor formation, growth and propagation, arteriosclerosis, ocular diseases, such as age-induced macular degeneration, choroidal neovascularisation and diabetic retinopathy, inflammatory diseases, arthritis, thrombosis, fibrosis, glomerulonephritis, neurodegeneration, psoriasis, restenosis, wound healing, transplant rejection, metabolic diseases, autoimmune diseases, cirrhosis, diabetes and vascular and immune diseases in mammals. The method includes administering to a subject in need thereof a therapeutically effective amount of a compound of any of the Formulae I-IV or a pharmaceutically acceptable salt, hydrate, prodrug, tautormer, enantiomer, or racemic mix thereof:

Subjects for treatment according to the present invention include humans (patients) and other mammals in need of therapy for the stated condition.

Compounds of the invention possess unique pharmacological characteristics with respect to inhibition of cellular division and influence the activity of the Aurora kinase enzymes in cells. Therefore, these compounds are effective in treating conditions and disorders, especially cancer-related tumors and disorders, which are modulated by Aurora kinase activity. In one embodiment, compounds of the invention are associated with diminished side effects compared to other current standards of treatment.

Compounds of the invention are typically more selective than known anti-cancer drugs, and demonstrate higher selectivity for inhibiting Aurora kinase activity. The compounds also exhibit an advantageous profile of activity including good bioavailability. Accordingly, they offer advantages over many art-known methods for treating disorders associated with unregulated or disturbed Aurora kinase activity.

### IV. General Syntheses

The compounds of the invention are prepared in general by methods known to those of skill in the art for synthesizing analogous compounds. These are illustrated by the general schemes indicated below, and the preparative examples that follow. Most

starting materials are commercially available from supply companies like Aldrich Chemicals Co. or Sigma Chemical Company, as examples. Compounds that are not commercially available may be synthesized by those of skill in the art by following procedures given in references such as “Organic Reactions,” Volumes 1-40, John Wiley & Sons (1991); “Rodd's Chemistry of Carbon Compounds,” Volumes 1-5 and Suppl., Elservier Science Publishers (1989); “Fieser and Fieser's Reagents for Organic Synthesis,” Volume 1-15, John Wiley & Sons (1991); and “Advanced Organic Chemistry,” Jerry March, John Wiley & Sons, 4^{th} Ed. (1992).

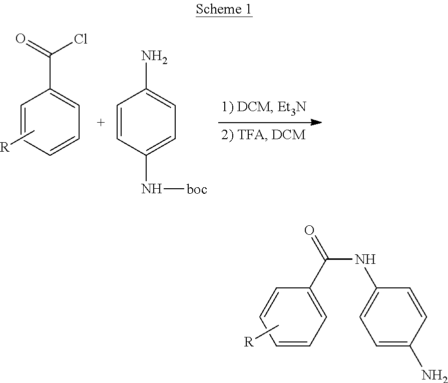

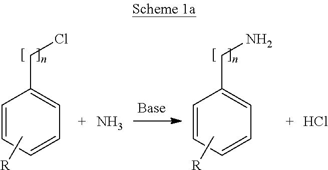

wherein R is as defined herein for Formula (I).

The product from Scheme 1 or Scheme 1a is used in the following synthesis of Scheme 2 based upon synthetic methods contained in WO 05/056552 to Vertex Pharmaceuticals Incorporated, the content of which is incorporated herein by reference.

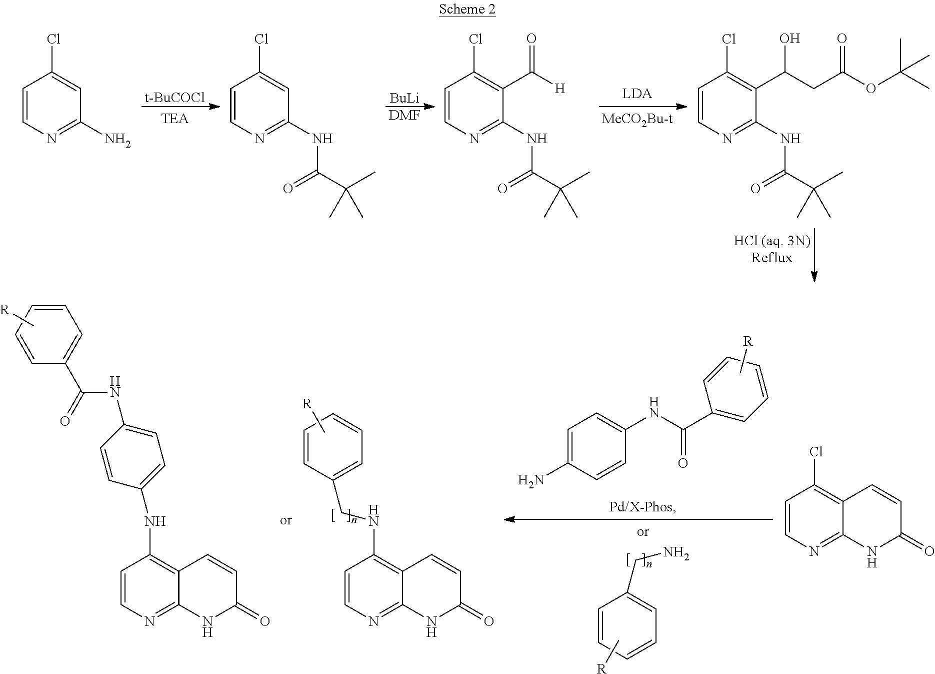

wherein:

R is H, halo, cyano, nitro, alkyl, trifluoromethyl, heteroalkyl, OR′, SR′ and NR′R″, where R′ and R″ each independently are H, alkyl, haloalkyl, alkylhalo, or heteroalkyl; or R is an heteroalkyl chain that optionally is bound at either end to adjoining carbon atoms of the phenyl ring to which it is attached, thereby forming a bicyclic ring structure; and

n is 1, 2, 3 or 4, with the proviso that n may=0 when X is other than oxygen.

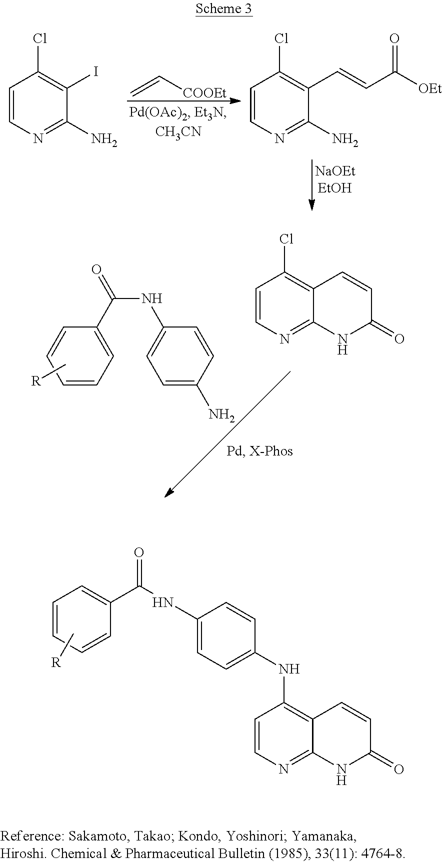

An alternative synthetic route for preparing compounds of the present invention is given in the following Scheme 3:

wherein: R is as defined above for Formula (I).

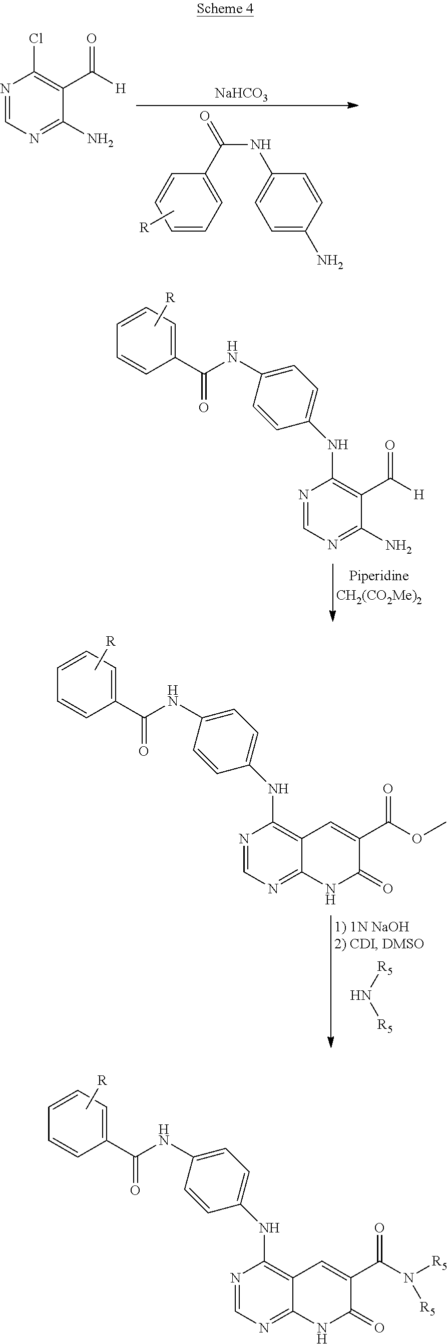

wherein: R is as defined in Formulae I-IV given above, and R₅ is as defined for

R₂, R₃ and in Formulae I-IV above.

The heterocyclic inhibitor/agonists of the invention are characterized by a core-moiety comprising a naphthyridine core. In an exemplary embodiment, the core-moity includes a naphthyridine heterocyclic ring system that further is substituted at the 3-position by a double bonded heteroatom and at the 6-position by a ligand bound chain containing at least one additional aryl or heterocyclic moiety. A preferred aryl moiety is substituted or unsubstituted phenyl group, and exemplary heterocyclic moieties include rings such as piperazinyl, piperidinyl, benzodioxolinyl, furanyl, benzofuranyl, oxazolyl, isoxazolyl, thiazolyl, isothiazolyl, imidazolyl and pyrazolyl groups.

The following examples are provided to illustrate selected embodiments of the invention and are not to be construed as limiting its scope.

### EXAMPLES

### Example 1

### N-(4-Aminophenyl)-2-fluoro-benzamide

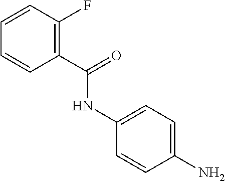

To a solution of N-Boc-1,4-phenylenediamine (1.0 g) in DCM (10 mL) was added triethylamine (1.1 eq., 971.78 mg) and 2-fluorobenzoyl chloride (1.1 eq., 837.56 mg). A precipitate was formed after 30 minutes stirring. The precipitate (1.5 g) was filtered and dried. It was suspended in DCM (5 mL) and trifluoroacetic acid (15 mL), and stirred for 15 minutes. The solvents were removed, and the residue was dissolved in EtOAc/water and the pH was adjusted to 10 with potassium carbonate solution. The EtOAc layer was separated and the aq. layer was extracted with more EtOAc. The combined organic layer was washed with water once, dried over MgSO4. The product (720 mg) obtained after concentration was used for the next reaction without further purification. LCMS [231.2 (M+1)].

### Example 2

### N-(5-Amino-pyrimidin-2-yl)-benzamide

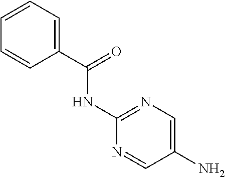

To a solution of 2-amino-5-nitropyrimidine (1.0 g) in DCM (10 mL) was added triethylamine (1.1 eq) and benzoyl chloride (1.1 eq.). The precipitate (1.1 g) that obtained after 30 minutes was filtered and dried. The solid was dissolved in MeOH (30 mL) and hydrogenated overnight at 30 psi in the presence of Pd/C (100 mg). The catalyst was filtered through a pad of Celite. MeOH was removed and the crude product (820 mg) was used for the next reaction without further purification. LCMS [215.2 (M+1)].

### Example 3

### N-(4-Aminophenyl)-3-fluoro-benzamide

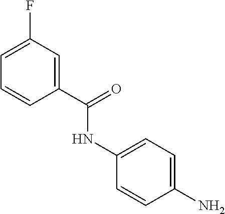

The title compound (910 mg) was synthesized according to the procedure described for the preparation of Example 1 by using N-Boc-1,4-phenylenediamine (1.0 g), 1.1 eq. of triethylamine and 3-benzoyl chloride (1.1 eq). LCMS [231.2 (M+1)].

### Example 4

### N-(4-Aminophenyl)-2-trifluoromethyl-benzamide

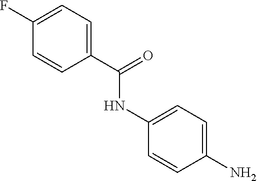

The title compound (880 mg) was synthesized according to the procedure described for the preparation of Example 1 with N-Boc-1,4-phenylenediamine (1.0 g), 1.1 eq. of triethylamine and 4-benzoyl chloride (1.1 eq). LCMS [231.2 (M+1)].

### Example 5

### N-(4-Aminophenyl)-2-trifluoromethyl-benzamide

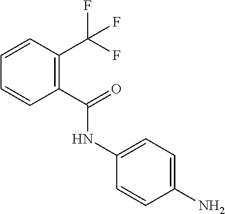

The title compound (920 m) was synthesized according to the procedure described for the preparation of Example 1 with N-Boc-1,4-phenylenediamine (1.0 g), 1.1 eq. of triethylamine and 2-trifluoromethyl-benzoyl chloride (1.1 eq). LCMS [281.25 (M+1)].

### Example 6

### N-(4-Aminophenyl)-4-trifluoromethyl-benzamide

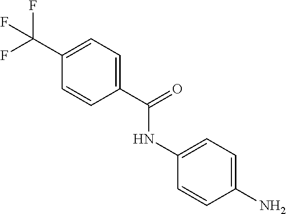

The title compound (900 mg) was synthesized according to the procedure described for the preparation of Example 1 with N-Boc-1,4-phenylenediamine (1.0 g), 1.1 eq. of triethylamine and 4-trifluoromethyl-benzoyl chloride (1.1 eq). LCMS [281.25 (M+1)].

### Example 7

### N-(4-Aminophenyl)-2-fluoro-3-trifluoromethyl-benzamide

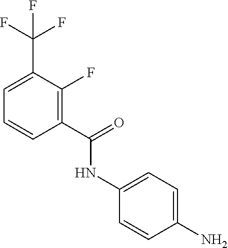

The title compound (830 mg) was synthesized according to the procedure described for the preparation of Example 1 with the starting materials of N-Boc-1,4-phenylenediamine (1.0 g) and 2-fluoro-3-trifluoromethyl-benzoyl chloride (1.1 eq). LCMS [299.2 (M+1)].

### Example 8

### N-(4-Aminophenyl)-4-fluoro-2-trifluoromethyl-benzamide

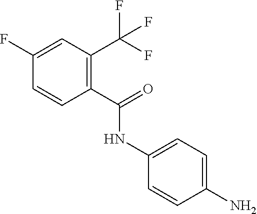

The title compound (900 mg) was synthesized according to the procedure described for the preparation of Example 1 with N-Boc-1,4-phenylenediamine (1.0 g), 1.1 eq. of triethylamine and 4-fluoro-2-trifluoromethyl-benzoyl chloride (1.1 eq). LCMS [299.2 (M+1)].

### Example 9

### N-(4-Aminophenyl)-2,6-difluoro-benzamide

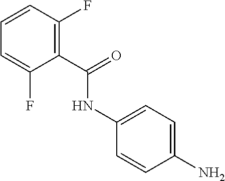

The title compound (820 mg) was synthesized according to the procedure described for the preparation of Example 1 with N-Boc-1,4-phenylenediamine (1.0 g), 1.1 eq. of triethylamine and 2,6-difluoro-benzoyl chloride (1.1 eq). LCMS [249.2 (M+1)].

### Example 10

### N-(4-Aminophenyl)-3,4-difluoro-benzamide

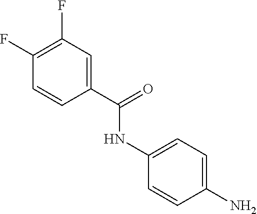

The title compound (850 mg) was synthesized according to the procedure described for the preparation of Example 1 with N-Boc-1,4-phenylenediamine (1.0 g), 1.1 eq. of triethylamine and 3,4-difluorobenzoyl chloride (1.1 eq). LCMS [249.2 (M+1)].

### Example 11

### N-(4-Aminophenyl)-3,5-difluoro-benzamide

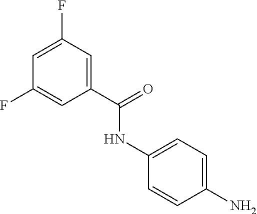

The title compound (720 mg) was synthesized according to the procedure described for the preparation of Example 1 with the starting materials of N-Boc-1,4-phenylenediamine (1.0 g), 1.1 eq. of triethylamine and 3,5-difluoro-benzoyl chloride (1.1 eq). LCMS [249.2 (M+1)].

### Example 12

### N-(4-Aminophenyl)-2,4-difluoro-benzamide

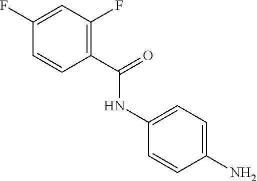

The title compound (810 mg) was synthesized according to the procedure described for the preparation of Example 1 with N-Boc-1,4-phenylenediamine (1.0 g), 1.1 eq. of triethylamine and 2,4-difluoro-benzoyl chloride (1.1 eq). LCMS [249.2 (M+1)].

### Example 13

### Cyclohexanecarboxylic acid (4-aminophenyl)-amide

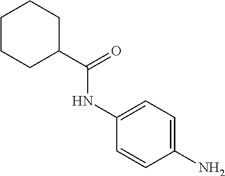

The title compound (550 mg) was synthesized according to the procedure described for the preparation of Example 1 with N-Boc-1,4-phenylenediamine (1.0 g), triethylamine (1.1 eq) and cyclohexyl chloride (1.1 eq). LCMS [219.3 (M+1)].

### Example 14

### N-(4-Aminophenyl)-3,5-bis-trifluoromethyl-benzamide

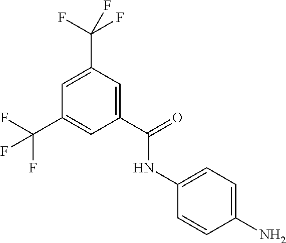

The title compound (600 mg) was synthesized according to the procedure described for the preparation of Example 1 with N-Boc-1,4-phenylenediamine (1.0 g), triethylamine (1.1 eq) and 3,5-bis-trifluoromethyl-benzoyl chloride (1.1 eq). LCMS [349.2 (M+1)].

### Example 15

### Naphthalene-2-carboxylic acid (4-aminophenyl)-amide

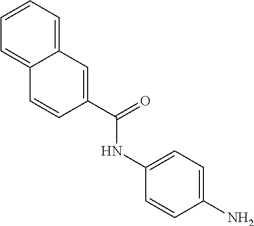

The title compound (700 mg) was synthesized according to the procedure described for the preparation of Example 1 with N-Boc-1,4-phenylenediamine (1.0 g), triethylamine (1.1 eq) and 2-naphthoyl chloride (1.1 eq). LCMS [263.1 (M+1)].

### Example 16

### N-(4-Aminophenyl)-2-methoxy-benzamide

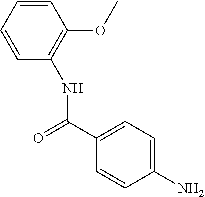

The title compound (750 mg) was synthesized according to the procedure described for the preparation of Example 1 with N-Boc-1,4-phenylenediamine (1.0 g), triethylamine (1.1 eq) and 2-methoxy-benzoyl chloride (1.1 eq). LCMS [243.2 (M+1)].

### Example 17

### N-(4-Aminophenyl)-4-methyl-benzamide

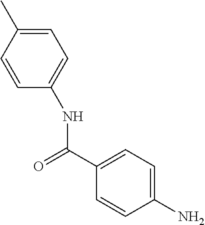

The title compound (700 mg) was synthesized according to the procedure described for the preparation of Example 1 with N-Boc-1,4-phenylenediamine (1.0 g), triethylamine (1.1 eq) and 4-methylbenzoyl chloride (1.1 eq). LCMS [227.2 (M+1)].

### Example 18

### N-(4-Aminophenyl)-2-fluoro-4-trifluoromethyl-benzamide

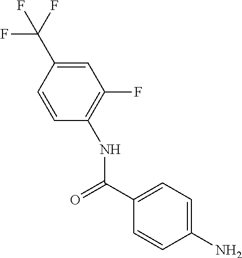

The title compound (890 mg) was synthesized according to the procedure described for the preparation of Example 1 with N-Boc-1,4-phenylenediamine (1.0 g), triethylamine (1.1 eq) and 2-fluoro-4-trifluoromethyl-benzoyl chloride (1.1 eq). LCMS [299.2 (M+1)].

### Example 19

### N-(4-Aminophenyl)-3-fluoro-5-trifluoromethyl-benzamide

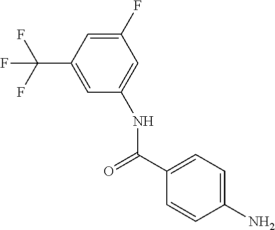

The title compound (880 mg) was synthesized according to the procedure described for the preparation of Example 1 with N-Boc-1,4-phenylenediamine (1.0 g), triethylamine (1.1 eq) and 3-fluoro-5-trifluoromethyl-benzoyl chloride (1.1 eq). LCMS [299.2 (M+1)].

### Example 20

### N-(4-Aminophenyl)-4-chloro-benzamide

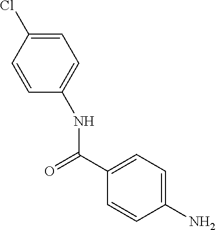

The title compound (780 mg) was synthesized according to the procedure described for the preparation of Example 1 with N-Boc-1,4-phenylenediamine (1.0 g), triethylamine (1.1 eq) and 4-chloro-benzoyl chloride (1.1 eq). LCMS [247.6 (M+1)].

### Example 21

### N-(4-Aminophenyl)-4-trifluoromethoxy-benzamide

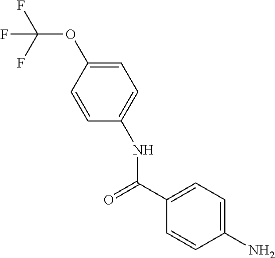

The title compound (750 mg) was synthesized according to the procedure described for the preparation of Example 1 with N-Boc-1,4-phenylenediamine (1.0 g), triethylamine (1.1 eq) and 4-trifluoromethoxybenzoyl chloride (1.1 eq). LCMS [297.2 (M+1)].

### Example 22

### N-(4-Aminophenyl)-2-methyl-benzamide

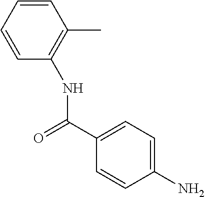

The title compound (700 mg) was synthesized according to the procedure described for the preparation of Example 1 with N-Boc-1,4-phenylenediamine (1.0 g), triethylamine (1.1 eq) and 2-methyl-benzoyl chloride (1.1 eq). LCMS [227.2 (M+1)].

### Example 23

### N-(4-Aminophenyl)-3-methyl-benzamide

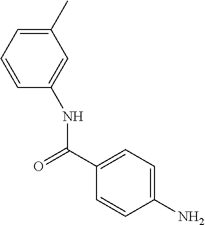

The title compound (710 mg) was synthesized according to the procedure described for the preparation of Example 1 with N-Boc-1,4-phenylenediamine (1.0 g), triethylamine (1.1 eq) and 3-methyl-benzoyl chloride (1.1 eq). LCMS [227.2 (M+1)].

### Example 24

### Naphthalene-1-carboxylic acid (4-aminophenyl)-amide

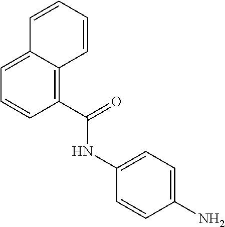

The title compound (750 mg) was synthesized according to the procedure described for the preparation of Example 1 with N-Boc-1,4-phenylenediamine (1.0 g), triethylamine (1.1 eq) and 1-naphthoyl chloride (1.1 eq). LCMS [263.1 (M+1)].

### Example 25

### N-(4-Aminophenyl)-2,6-dichloro-benzamide

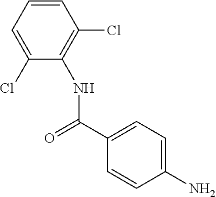

The title compound (940 mg) was synthesized according to the procedure described for the preparation of Example 1 with N-Boc-1,4-phenylenediamine (1.0 g), triethylamine (1.1 eq) and 2,6-dichlorobenzoyl chloride (1.1 eq). LCMS [281.1 (M+1)].

### Example 26

### N-(4-Aminophenyl)-3,4-dichloro-benzamide

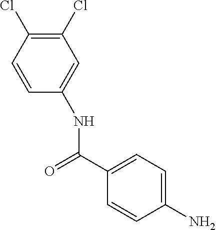

The title compound (890 mg) was synthesized according to the procedure described for the preparation of Example 1 with N-Boc-1,4-phenylenediamine (1.0 g), triethylamine (1.1 eq) and 3,4-dichlorobenzoyl chloride (1.1 eq). LCMS [281.1 (M+1)].

### Example 27

### N-(4-Aminophenyl)-2,4-dichloro-benzamide

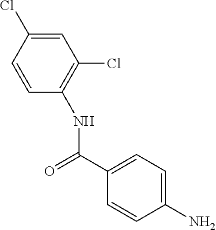

The title compound (890 mg) was synthesized according to the procedure described for the preparation of Example 1 with N-Boc-1,4-phenylenediamine (1.0 g), triethylamine (1.1 eq) and 2,4-dichlorobenzoyl chloride (1.1 eq). LCMS [282.1 (M+1)].

### Example 28

### N-(4-chloropyridin-2-yl)-2,2-dimethylpropanamide

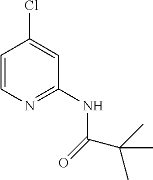

To a solution of 4-chloropyridin-2-amine (8.30 g; 64.56 mmol; 1.00 eq.) in DCM/Pyridine (100/100 mL) was added N,N-diethylethanamine (8.17 g; 80.70 mmol; 1.25 eq.), followed with 2,2-dimethylpropanoyl chloride (8.56 g; 71.02 mmol; 1.10 eq.). The mixture was stirred overnight. After removal of the solvents, the residue was purified by silica gel flash chromatography to afford N-(4-chloropyridin-2-yl)-2,2-dimethylpropanamide (11 g). 1H NMR (400 MHz, DMSO-D6): 1.23 (s, 9H), 7.24 (d, J=1.6 Hz, 1H), 8.17 (s, 1H), 8.32 (d, J=1.6 Hz, 1H), 10.25 (s, 1H).

### Example 29

### N-(4-chloro-3-formylpyridin-2-yl)-2,2-dimethylpropanamide

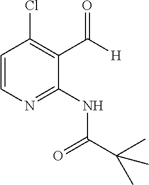

To a solution of N-(4-chloropyridin-2-yl)-2,2-dimethylpropanamide (3.30 g; 15.52 mmol; 1.00 eq.) in THF (40 mL) at −78° C. was added butyllithium (15.52 ml; 2.50 M; 38.79 mmol; 2.50 eq.) dropwise. The mixture was stirred for 30 minutes, and a solution of N,N-dimethylformamide (4.40 ml; 46.55 mmol; 3.00 eq.) in THF (10 mL) was added. After 1 h at −78° C., the mixture was warmed to room temperature. Saturated NH4Cl (100 mL) was added, and stiffing continued for 30 minutes. The solution was extracted with EtOAc, dried over MgSO₄. The solvent was evaporated, and the residue was purified through flash chromatography on silica gel to provide 2.1 g of the desired product. ¹H NMR (400 MHz, DMSO-D₆): 1.24 (s, 9H), 7.48 (d, J=1.6 Hz, 1H), 8.52 (d, J=1.6 Hz, 1H), 9.96 (s, 1H), 10.80 (s, 1H).

### Example 30

### Tert-butyl 3-{4-chloro-2-[(2,2-dimethylpropanoyl)amino]pyridin-3-yl}-3-hydroxypropanoate

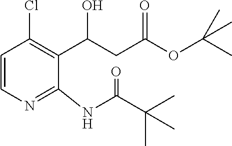

To a solution of N-isopropylpropan-2-amine (4.17 ml; 29.52 mmol; 2.20 eq.) in THF (30 mL) at 0° C. was added butyllithium (11.81 ml; 2.50 M; 29.52 mmol; 2.20 eq.). The mixture was stirred for 10 minutes and cooled to −78° C. Tert-butyl acetate (3.98 ml; 29.52 mmol; 2.20 eq.) in THF (5 mL) was added drop wise to the above solution and after 15 minutes, a solution of N-(4-chloro-3-formylpyridin-2-yl)-2,2-dimethylpropanamide (3.23 g; 13.42 mmol; 1.00 eq.) in THF (15 mL) was added at this temperature. After stirring for 30 minutes, the mixture was warmed to rt, and poured into water. Extracted with ether, dried over MgSO₄, and concentrated the ethereal layer. The residue was purified through flash chromatography on silica gel to obtain 2.0 g of the desired product. LCMS: 357.75 (M+H).

### Example 31

### 5-Chloro-1,8-naphthyridi-2(1H)-one

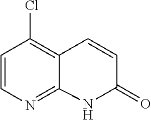

A solution of tert-butyl 3-{4-chloro-2-[(2,2-dimethylpropanoyl)-amino]pyridin-3-yl}-3-hydroxypropanoate (3.50 g; 5.60 mmol) in aqueous hydrogen chloride (40.00 ml; 3.00 M; 75.00 mmol) was refluxed for 1.5 h (Continuous monitoring of reaction is necessary, because prolonged heating will result in by product). Cooling to rt gave a precipitate, which was filtered, washed with sat. NaHCO₃, water and dried. The filtrate was refluxed again for 30 minutes, and obtained more compound was filtered after cooling to rt. Heating of the filtrate and cooling followed by filtration was repeated several times to get a combined product of 1.9 g, which was washed with water and dried. The crude was used as such for the next reaction. LCMS [181 (M+1)]. ¹H NMR (400 MHz, CD₃OD): 6.71 (d, J=3.4 Hz, 1H), 7.42 (d, J=1.5 Hz, 1H), 8.06 (d, J=3.4 Hz, 1H), 8.47 (d, J=1.5 Hz, 1H).

### Example 31a

### 5-(5-chlorobenzo[d][1,3]dioxo-4-ylamino)-1,8-naphthyridin-2(1H)-one

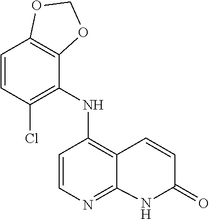

### Example 32

### N-(4-(7-oxo-7,8-dihydro-1,8-naphthyridin-4-yl-amino)phenyl)benzamide

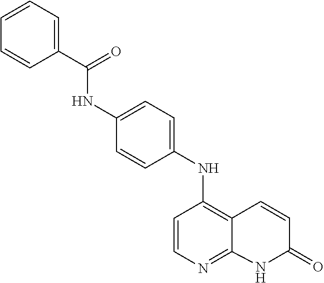

The suspension of 5-Chloro-1,8-naphthyridi-2(1H)-one (100 mg), 4′-aminobenzanilide (150 mg), allylchloro[1,3-bis(2,6-di-iso-propylphenyl)imidazol-2-ylidene]palladium (II) (12.70 mg), 2-dicyclohexylphosphino-2′-4′-6′-triisopropylbiphenyl (21.12 mg) and sodium tert-butoxide (212.86 mg) in dioxane (3 mL) was stirred at 100° C. for 24 h in sealed tube. After cooling to room temperature, the solid was filtered, washed with water and methanol and dried. 20 mg of the desired product was obtained. LCMS [357.3 (M+1)].

### Example 33

### N-(4-(7-oxo-7,8-dihydro-1,8-naphthyridin-4-yl-amino)pyrimidin-2-yl)benzamide

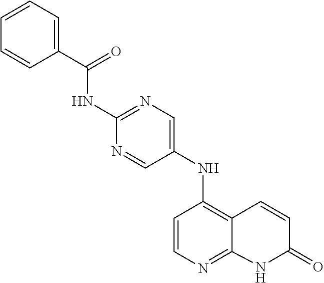

N-(4-(7-oxo-7,8-dihydro-1,8-naphthyridin-4-yl-amino)pyrimidin-2-yl)benzamide was synthesized according to the procedure described for the preparation of Example 32. LCMS [359.3 (M+1)].

### Example 34

### 2-Fluoro-N-(4-(7-oxo-7,8-dihydro-1,8-naphthyridin-4-yl-amino)phenyl)benzamide

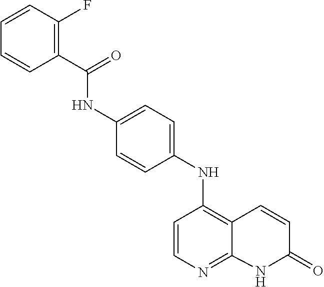

2-Fluoro-N-(4-(7-oxo-7,8-dihydro-1,8-naphthyridin-4-yl-amino)phenyl)benzamide was synthesized according to the procedure described for the preparation of Example 32. LCMS [375.3 (M+1)].

### Example 35

### 3-Fluoro-N-(4-(7-oxo-7,8-dihydro-1,8-naphthyridin-4-yl-amino)phenyl)benzamide

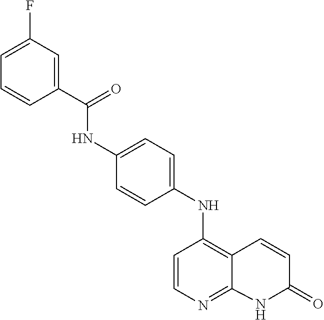

3-Fluoro-N-(4-(7-oxo-7,8-dihydro-1,8-naphthyridin-4-yl-amino)phenyl)benzamide was synthesized according to the procedure described for the preparation of Example 32. LCMS [375.3 (M+1)].

### Example 36

### 4-Fluoro-N-(4-(7-oxo-7,8-dihydro-1,8-naphthyridin-4-yl-amino)phenyl)benzamide

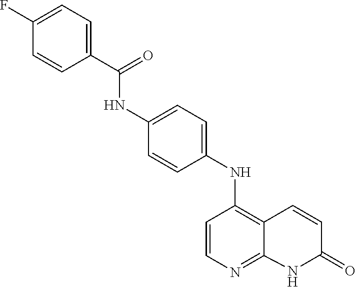

4-Fluoro-N-(4-(7-oxo-7,8-dihydro-1,8-naphthyridin-4-yl-amino)phenyl)benzamide was synthesized according to the procedure described for the preparation of Example 32. LCMS [375.3 (M+1)].

### Example 37

### 2-Trifluoromethyl-N-(4-(7-oxo-7,8-dihydro-1,8-naphthyridin-4-yl-amino)phenyl)benzamide

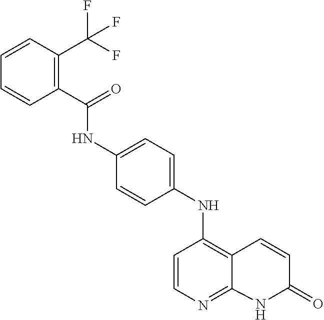

2-Trifluoromethyl-N-(4-(7-oxo-7,8-dihydro-1,8-naphthyridin-4-yl-amino)phenyl)benzamide was synthesized according to the procedure described for the preparation of Example 32. LCMS [425.3 (M+1)].

### Example 38

### 4-Trifluoromethyl-N-(4-(7-oxo-7,8-dihydro-1,8-naphthyridin-4-yl-amino)phenyl)benzamide

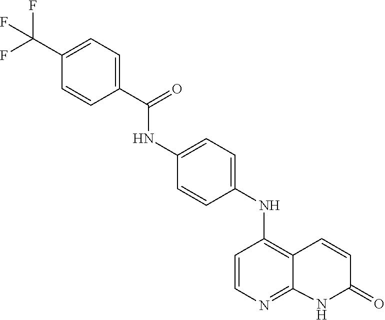

4-Trifluoromethyl-N-(4-(7-oxo-7,8-dihydro-1,8-naphthyridin-4-yl-amino)phenyl)benzamide was synthesized according to the procedure described for the preparation of Example 32. LCMS [425.3 (M+1)].

### Example 39

### 2-Fluoro-3-Trifluoromethyl-N-(4-(7-oxo-7,8-dihydro-1,8-naphthyridin-4-yl-amino)phenyl)benzamide

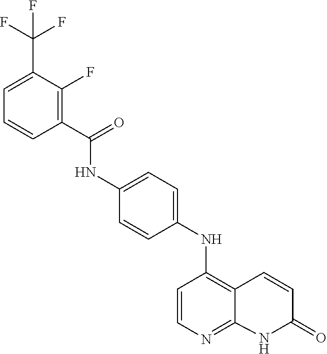

2-Fluoro-4-Trifluoromethyl-N-(4-(7-oxo-7,8-dihydro-1,8-naphthyridin-4-yl-amino)phenyl)benzamide was synthesized according to the procedure described for the preparation of Example 32. LCMS [443.3 (M+1)].

### Example 40

### 4-Fluoro-2-trifluoromethyl-N-(4-(7-oxo-7,8-dihydro-1,8-naphthyridin-4-yl-amino)phenyl)benzamide

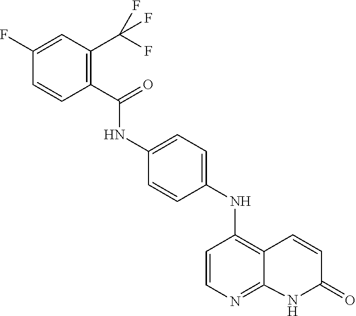

4-Fluoro-2-trifluoromethyl-N-(4-(7-oxo-7,8-dihydro-1,8-naphthyridin-4-yl-amino)phenyl)benzamide was synthesized according to the procedure described for the preparation of Example 32. LCMS [443.3 (M+1)].

### Example 41

### 2,6-Difluoro-N-(4-(7-oxo-7,8-dihydro-1,8-naphthyridin-4-yl-amino)phenyl)benzamide

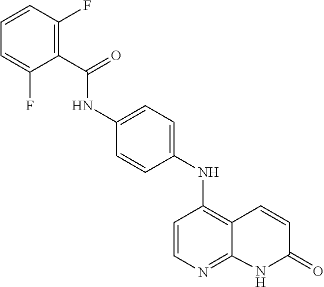

2,6-Difluoro-N-(4-(7-oxo-7,8-dihydro-1,8-naphthyridin-4-yl-amino)phenyl)benzamide was synthesized according to the procedure described for the preparation of Example 32. LCMS [393.3 (M+1)].

### Example 42

### 3,4-Difluoro-N-(4-(7-oxo-7,8-dihydro-1,8-naphthyridin-4-yl-amino)phenyl)benzamide

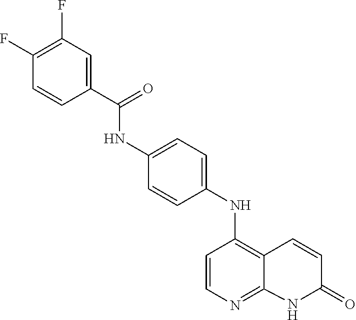

3,4-Difluoro-N-(4-(7-oxo-7,8-dihydro-1,8-naphthyridin-4-yl-amino)phenyl)benzamide was synthesized according to the procedure described for the preparation of Example 32. LCMS [393.3 (M+1)].

### Example 43

### 3,5-Difluoro-N-(4-(7-oxo-7,8-dihydro-1,8-naphthyridin-4-yl-amino)phenyl)benzamide

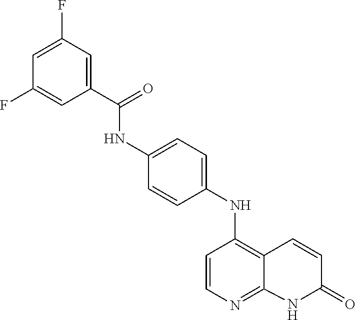

3,5-Difluoro-N-(4-(7-oxo-7,8-dihydro-1,8-naphthyridin-4-yl-amino)phenyl)benzamide was synthesized according to the procedure described for the preparation of Example 32. LCMS [393.3 (M+1)].

### Example 44

### 2,4-Difluoro-N-(4-(7-oxo-7,8-dihydro-1,8-naphthyridin-4-yl-amino)phenyl)benzamide

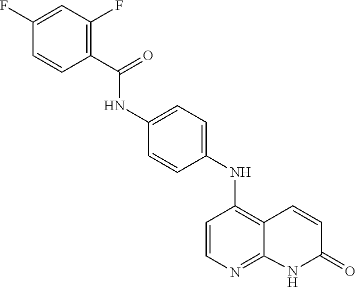

2,4-Difluoro-N-(4-(7-oxo-7,8-dihydro-1,8-naphthyridin-4-yl-amino)phenyl)benzamide was synthesized according to the procedure described for the preparation of Example 32. LCMS [393.3 (M+1)].

### Example 45

### N-(4-(7-oxo-7,8-dihydro-1,8-naphthyridin-4-yl-amino)phenyl)cyclohexanecarboxamide

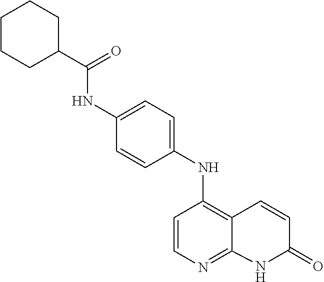

N-(4-(7-oxo-7,8-dihydro-1,8-naphthyridin-4-yl-amino)phenyl)cyclohexanecarboxamide was synthesized according to the procedure described for the preparation of Example 32. LCMS [363.3 (M+1)].

### Example 46

### N-(4-(7-oxo-7,8-dihydro-1,8-naphthyridin-4-yloxy)phenyl)benzamide

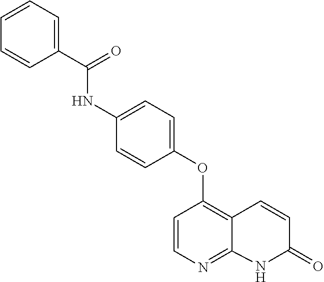

1-chlorobenzo[c]-1,8-naphthyridin-6(5H)-one (100.00 mg; 0.55 mmol; 1.00 eq.) was suspended in methanol (20 ml), and 1N HCl ether (1.0 N, 1.1 ml) was added. The reaction mixture was stirred at rt for overnight. The resulting HCl salt was filtered and dried. The HCl salt and N-(4-hydroxyphenyl)benzamide (141.69 mg; 0.66 mmol; 1.20 eq.), were dissolved in NMP (3 mL) and heated at 150° C. with stirring overnight. After cooling to rt, water (20 mL) was added and the precipitate was collected by filtration, washed with MeOH, and dried. 17 mg of N-(4-(7-oxo-7,8-dihydro-1,8-naphthyridin-4-yloxy)phenyl)benzamide was obtained. LCMS [358.3 (M+1)].

### Example 47

### N-(2-(7-oxo-7,8-dihydro-1,8-naphthyridin-4-ylamino)phenyl)benzamide

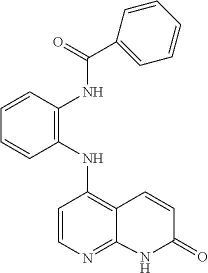

N-(2-(7-oxo-7,8-dihydro-1,8-naphthyridin-4-yl-amino)phenyl)benzamide was synthesized according to the procedure described for the preparation of Example 32. LCMS [357.3 (M+1)].

### Example 48

### 4-(7-Oxo-7,8-dihydro-1,8-naphthyridin-4-ylamino)-N-phenyl-benzamide

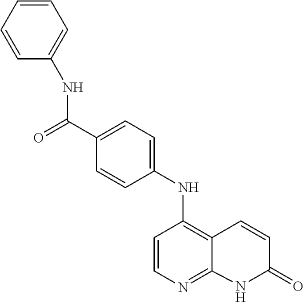

4-(7-Oxo-7,8-dihydro-1,8-naphthyridin-4-yl-amino)-N-phenyl)benzamide was synthesized according to the procedure described for the preparation of Example 32. LCMS [357.3 (M+1)].

### Example 49

### 3,5-Bis(trifluoromethyl)-N-(4-(7-oxo-7,8-dihydro-1,8-naphthyridin-4-yl-amino)phenyl)benzamide

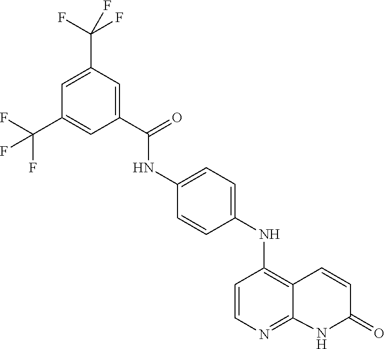

3,5-Bis(trifluoromethyl)-N-(4-(7-oxo-7,8-dihydro-1,8-naphthyridin-4-yl-amino)phenyl)benzamide was synthesized according to the procedure described for the preparation of Example 32. LCMS [493.3 (M+1)].

### Example 50

### N-Naphthalen-2-yl-4-(7-oxo-7,8-dihydro-1,8-naphthyridin-4-yl-amino)benzamide

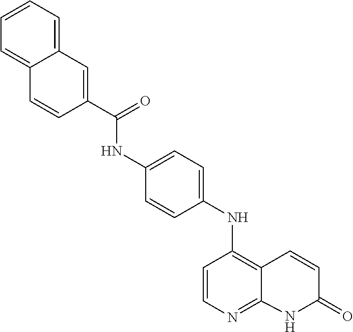

The title compound was synthesized according to the procedure described for the preparation of Example 32. LCMS [407.4 (M+1)].

### Example 51

### 2-Methoxy-N-(4-(7-oxo-7,8-dihydro-1,8-naphthyridin-4-yl-amino)phenyl)benzamide

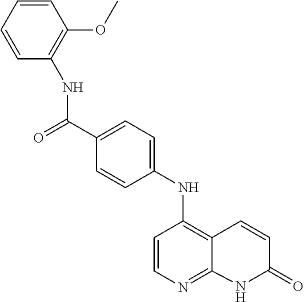

The title compound was synthesized according to the procedure described for the preparation of Example 32. LCMS [387.4 (M+1)].

### Example 52

### 4-Methyl-N-(4-(7-oxo-7,8-dihydro-1,8-naphthyridin-4-yl-amino)phenyl)benzamide

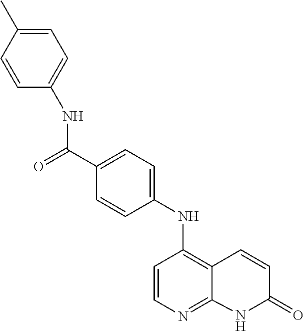

The title compound was synthesized according to the procedure described for the preparation of Example 32. LCMS [371.4 (M+1)].

### Example 53

### 2-Fluoro-4-trifluoromethyl-N-(4-(7-oxo-7,8-dihydro-1,8-naphthyridin-4-yl-amino)phenyl)benzamide

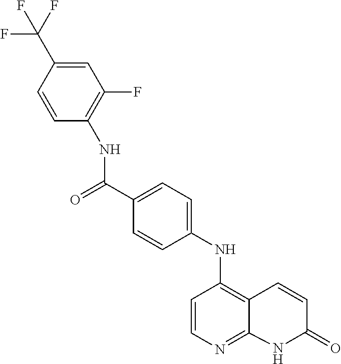

The title compound was synthesized according to the procedure described for the preparation of Example 32. LCMS [443.4 (M+1)].

### Example 54

### 3-Fluoro-5-trifluoromethyl-N-(4-(7-oxo-7,8-dihydro-1,8-naphthyridin-4-yl-amino)phenyl)benzamide

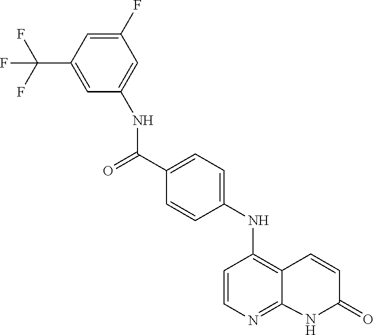

The title compound was synthesized according to the procedure described for the preparation of Example 32. LCMS [443.4 (M+1)].

### Example 55

### 4-Chloro-N-(4-(7-oxo-7,8-dihydro-1,8-naphthyridin-4-yl-amino)phenyl)benzamide

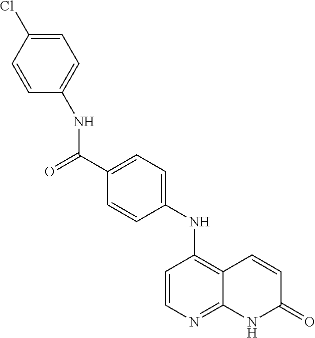

The title compound was synthesized according to the procedure described for the preparation of Example 32. LCMS [391.8 (M+1)].

### Example 56

### 4-Trifluoromethoxy-N-(4-(7-oxo-7,8-dihydro-1,8-naphthyridin-4-yl-amino)phenyl)benzamide

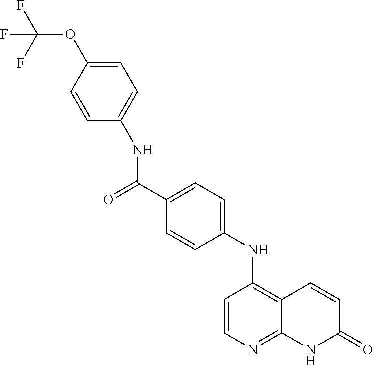

The title compound was synthesized according to the procedure described for the preparation of Example 32. LCMS [441.3 (M+1)].

### Example 57

### 2-Methyl-N-(4-(7-oxo-7,8-dihydro-1,8-naphthyridin-4-yl-amino)phenyl)benzamide

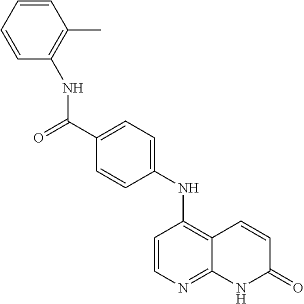

The title compound was synthesized according to the procedure described for the preparation of Example 32. LCMS [371.4 (M+1)].

### Example 58

### 3-Methyl-N-(4-(7-oxo-7,8-dihydro-1,8-naphthyridin-4-yl-amino)phenyl)benzamide

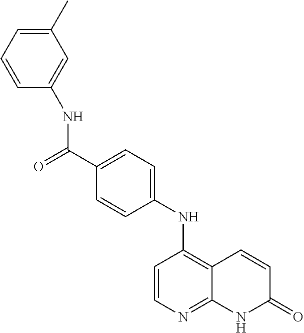

The title compound was synthesized according to the procedure described for the preparation of Example 32. LCMS [371.4 (M+1)].

### Example 59

### N-Naphthalen-1-yl-4-(7-oxo-7,8-dihydro-1,8-naphthyridin-4-yl-amino)benzamide

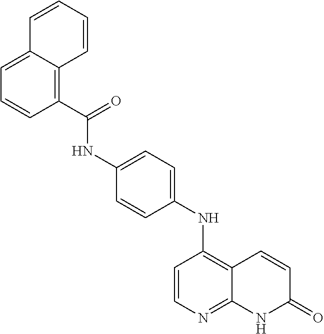

The title compound was synthesized according to the procedure described for the preparation of Example 32. LCMS [407.4 (M+1)].

### Example 60

### 2,6-Dichloro-N-(4-(7-oxo-7,8-dihydro-1,8-naphthyridin-4-yl-amino)phenyl)benzamide

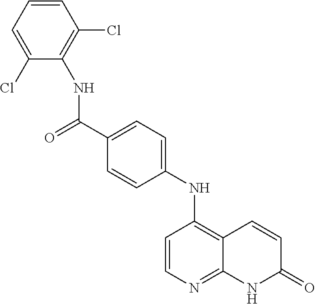

The title compound was synthesized according to the procedure described for the preparation of Example 32. LCMS [426.3 (M+1)].

### Example 61

### 3,4-Dichloro-N-(4-(7-oxo-7,8-dihydro-1,8-naphthyridin-4-yl-amino)phenyl)benzamide

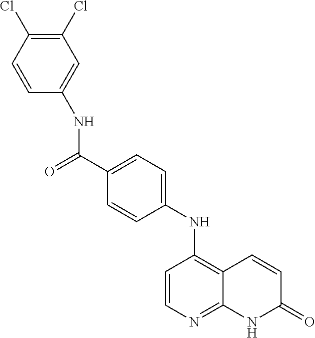

The title compound was synthesized according to the procedure described for the preparation of Example 32. LCMS [426.3 (M+1)].

### Example 62

### 2,4-Dichloro-N-(4-(7-oxo-7,8-dihydro-1,8-naphthyridin-4-yl-amino)phenyl)benzamide

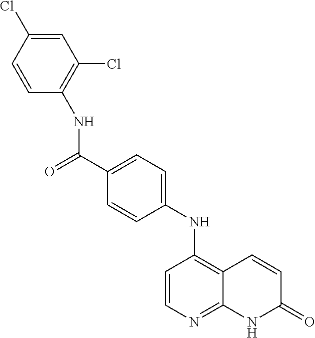

The title compound was synthesized according to the procedure described for the preparation of Example 32. LCMS [426.3 (M+1)].

### Example 63

### N-(4-(7-Chloro-1,8-naphthyridin-4-yl-amino)phenyl)benzamide

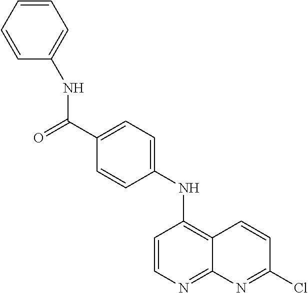

A suspension of N-(4-(7-oxo-7,8-dihydro-1,8-naphthyridin-4-yl-mino)phenyl)benzamide (1.2 g) in phosphorus oxychloride (30 mL) was stirred overnight at 100° C. After removal of the solvent, the crude product was used for the next reaction without further purification. LCMS [375.8 (M+1)].

### Example 64

### N-(4-(7(4-Dimethylamino-piperidin-1-yl-1,8-naphthyridin-4-yl-amino)phenyl)benzamide

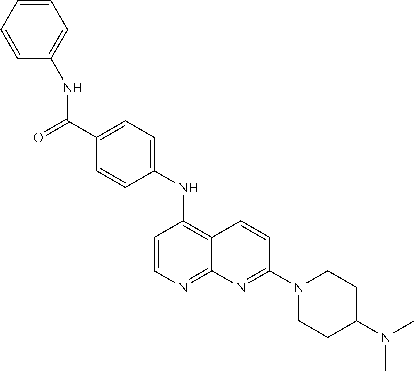

A suspension of N-{4-[(7-chloro-1,8-naphthyridin-4-yl)amino]phenyl}benzamide (100.00 mg; 0.27 mmol; 1.00 eq.) and N,N-dimethylpiperidin-4-amine (171.03 mg; 1.33 mmol; 5.00 eq.) in iPrOH (2 mL) was stirred at 100° C. for overnight. After cooling to rt, the title compound was obtained after purification using reverse-phase HPLC. LCMS [467.5 (M+1)].

### Example 65

### N-(4-(7-(4-Dimethylamino-ethylamino)-1,8-naphthyridin-4-yl-amino)phenyl)benzamide

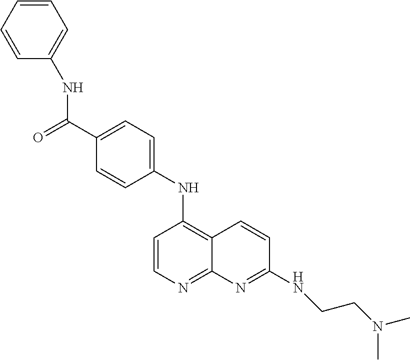

The title compound was synthesized according to the procedure described for the preparation of Example 64. LCMS [427.5 (M+1)].

### Example 66

### N-(4-(7-(4-Dimethylamino-propylamino)-1,8-naphthyridin-4-yl-amino)phenyl)benzamide

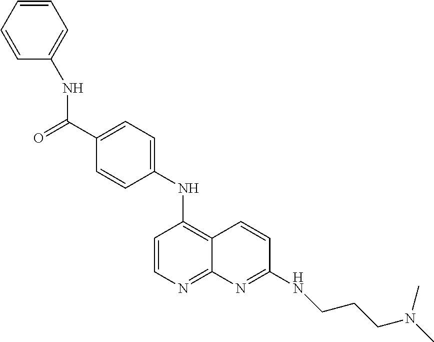

The title compound was synthesized according to the procedure described for the preparation of Example 64. LCMS [441.5 (M+1)].

### Example 67

### N-(4-(7-(4-Dimethylamino-pyrrolidin-1-yl)-1,8-naphthyridin-4-yl-amino)phenyl)benzamide

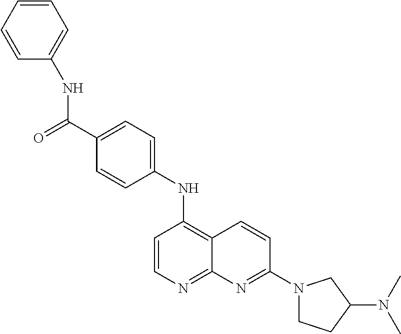

The title compound was synthesized according to the procedure described for the preparation of Example 64. LCMS [453.5 (M+1)].

### Example 68

### N-(4-(7-(4-Dimethylamino-ethoxy)-1,8-naphthyridin-4-yl-amino)phenyl)benzamide

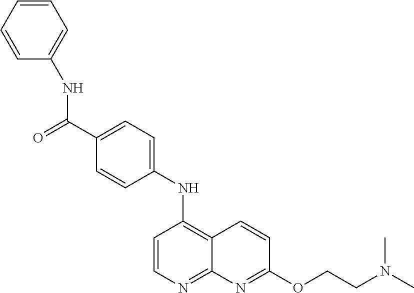

The title compound was synthesized according to the procedure described for the preparation of Example 64. LCMS [428.5 (M+1)].

### Example 69

### N-(4-(7-(4-Dimethylamino-propoxy)-1,8-naphthyridin-4-yl-amino)phenyl)benzamide

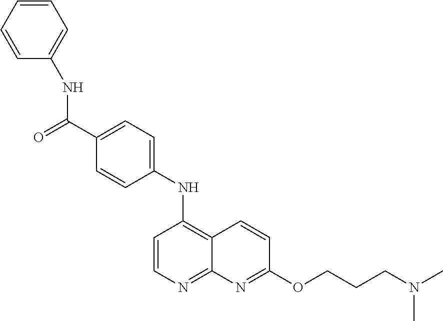

The title compound was synthesized according to the procedure described for the preparation of Example 64. LCMS [442.5 (M+1)].

### Example 70

### N-(4-(7-(2-pyrrolidin-1-yl-ethoxy)-1,8-naphthyridin-4-yl-amino)phenyl)benzamide

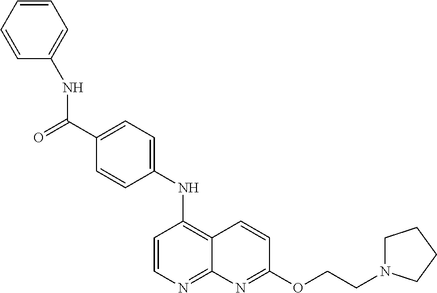

The title compound was synthesized according to the procedure described for the preparation of Example 64. LCMS [454.5 (M+1)].

### Example 71

### N-(4-(7-(3-morpholin-4-yl-propoxy)-1,8-naphthyridin-4-yl-amino)phenyl)benzamide

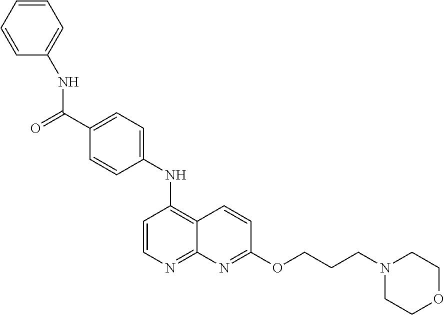

The title compound was synthesized according to the procedure described for the preparation of Example 64. LCMS [484.5 (M+1)].

### Example 72

### N-(4-(7-(2-morpholin-4-yl-ethoxy)-1,8-naphthyridin-4-yl-amino)phenyl)benzamide

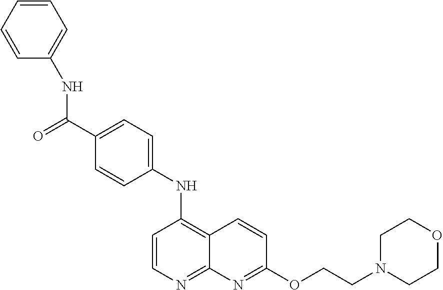

The title compound was synthesized according to the procedure described for the preparation of Example 64. LCMS [470.5 (M+1)].

### Example 73

### N-(3-Benzoyl-4-chloropyridin-2-yl)-2,2-dimethylpropanamide

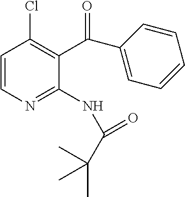

To a solution of N-(4-chloropyridin-2-yl)-2,2-dimethylpropanamide (4.00 g; 18.81 mmol 1.00 eq.) in THF (40 mL) at −78° C. was added butyllithium (15.80 ml; 2.50 M; 39.50 mmol; 2.10 eq.) dropwise. The mixture was stirred for 3 h at 0° C. and the solution was cooled to −78° C. again. N-methoxy-N-methylbenzamide (3.42 g; 20.69 mmol; 1.10 eq.) in THF (10 mL) was added. After 1 h at −78° C., the mixture was warmed to rt. The reaction was quenched with addition of water (10 mL), extracted with EtOAc, dried over MgSO4 and the EtOAc layer was concentrated. The residue was purified by silica gel flash chromatography to provide 1.3 g of N-(3-Benzoyl-4-chloropyridin-2-yl)-2,2-dimethylpropanamide. LCMS [317.7 (M+1)].

### Example 74

### (2-Amino-4-chloro-pyridin-3-yl)-phenyl-methanone

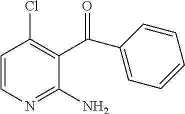

A suspension of N-(3-benzoyl-4-chloropyridin-2-yl)-2,2-dimethylpropanamide (400.00 mg; 1.26 mmol) in 10 mL of 3N aq.HCl was heated to reflux for overnight. After cooling to rt, the mixture was neutralized with 3N aq. NaOH, and extracted with DCM (4×20 mL). The residue was purified by silica gel flash chromatography to provide 210 mg of (2-Amino-4-chloro-pyridin-3-yl)-phenyl-methanone.

### Example 75

### 5-Chloro-2-oxo-4-phenyl-1,2-dihydro-1,8-naphthyridi-3-carboxylic acid tert-butyl ester

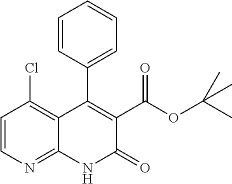

A mixture of (2-Amino-4-chloro-pyridin-3-yl)-phenyl-methanone (200 mg), di-tert-butyl malonate (2 mL) and potassium hydroxide (20 mg) was stirred at 150-170° C. under the protection of Argon gas for 5 h. The reaction was completed. The product was filtered and washed with water and MeOH to provide 300 mg of the 5-Chloro-2-oxo-4-phenyl-1,2-dihydro-1,8-naphthyridi-3-carboxylic acid tert-butyl ester. LCMS [357.7 (M+1)].

### Example 76

### 5-[(3,4-Difluoro-benzoylamino)-phenylamino]-2-oxo-4-phenyl1,2-dihydro-1,8-naphthyridine-3-carboxylic acid

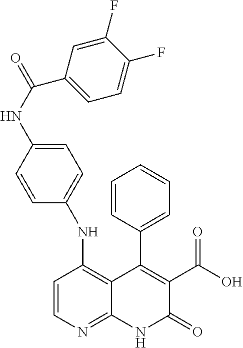

The title compound was synthesized according to the procedure described for the preparation of Example 32. LCMS [513.4 (M+1)].

### Example 77

### 5-[(3,4-Difluoro-benzoylamino)-phenylamino]-2-oxo-4-phenyl-1,2-dihydro-1,8-naphthyridine-3-carboxylic acid (2-dimethylamino-ethyl)-amide

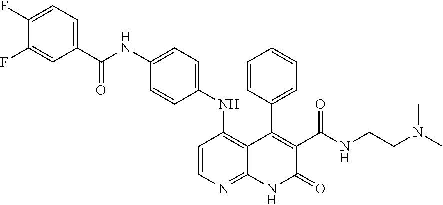

To a solution of 5-({4-[(3,4-difluorobenzoyl)amino]phenyl}amino)-2-oxo-4-phenyl-1,2-dihydro-1,8-naphthyridine-3-carboxylic acid (50.00 mg; 0.10 mmol; 1.00 eq.) in DMSO (2 mL) was added 1,1′-carbonylbis(1H-imidazole) (31.64 mg; 0.20 mmol; 2.00 eq.). The mixture was stirred for overnight. N,N-dimethylethane-1,2-diamine (25.80 mg; 0.29 mmol; 3.00 eq.) was added and the mixture was stirred for 5 h. The product, 5-[(3,4-Difluoro-benzoylamino)-phenylamino]-2-oxo-4-phenyl-1,2-dihydro-1,8-naphthyridine-3-carboxylic acid (2-dimethylamino-ethyl)-amide (3 mg) was obtained through purification with RP-HPLC. LCMS [583.5 (M+1)].

### Example 78

### 5-[(3,4-Difluoro-benzoylamino)-phenylamino]-2-oxo-4-phenyl-1,2-dihydro-1,8-naphthyridine-3-carboxylic acid (2-hydroxy-ethyl)-amide

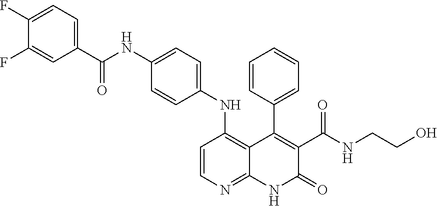

The title compound was synthesized according to the procedure described for the preparation of Example 77. LCMS [556.4 (M+1)].

### Example 79

### 5-[(3,4-Difluoro-benzoylamino)-phenylamino]-2-oxo-4-phenyl-1,2-dihydro-1,8-naphthyridine-3-carboxylic acid ((S)2,3-dihydroxypropyl)-amide

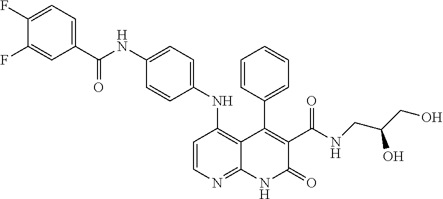

The title compound was synthesized according to the procedure described for the preparation of Example 77. LCMS [586.5 (M+1)].

### Example 80

### (5-Chloro-2-oxo-1,2-dihydro-1,8-naphthyridin-3-yl)-acetic acid

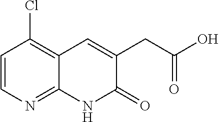

The title compound was synthesized according to the procedure described for the preparation of Example 31. LCMS [239.5 (M+1)].

### Example 81

### 5-[(3,4-Difluoro-benzoylamino)-phenylamino]-2-oxo-4-phenyl-1,2-dihydro-1,8-naphthyridine-3-acetic acid

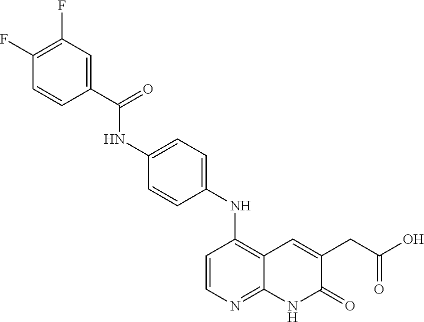

The title compound was synthesized according to the procedure described for the preparation of Example 32. LCMS [402.3 (M+1)].

### Example 82

### 5-[(3,4-Difluoro-benzoylamino)-phenylamino]-2-oxo-4-phenyl-1,2-dihydro-1,8-naphthyridine-3-acetic acid (2-hydroxy-ethyl)-amide

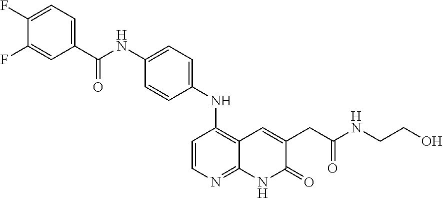

The title compound was synthesized according to the procedure described for the preparation of Example 77. LCMS [494.4 (M+1)].

### Example 83

### N-[4-(6-Amino-5-formyl-pyrimidin-4-ylamino)-phenyl]-benzamide

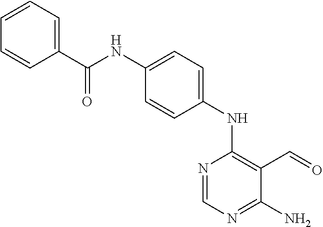

The mixture of 4-amino-6-chloro-pyrimidine-5-carbaldehyde (4.0 g, 25.39 mmol) and 4′-aminobenzanilide (6.47 g, 30.46 mmol, 1.2 eq.) and sodium bicarbonate (4.27 g, 50.77 mmol, 2.0 eq) in MeOH/water (100 ml/50 mL) was stirred at 60° C. for overnight. After cooling to room temperature, the solid (8.2 g) was filtered, washed with water and methanol and then dried under vacuum. LCMS [334.3 (M+1)].

### Example 84

### 4-(4-Benzoylamino-phenylamino)-7-oxo-7,8-dihydro-pyrido[2,3-d]pyrimidine-6-carboxylic acid methyl ester

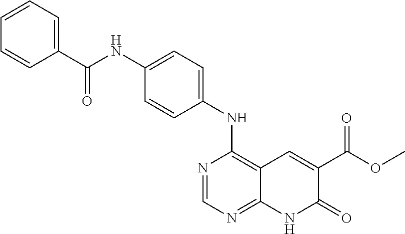

To a suspension of N-[4-(6-Amino-5-formyl-pyrimidin-4-ylamino)-phenyl]-benzamide (200 mg, 0.6 mmol) in ethanol (5 mL) was added dimethyl malonate (358.53 mg, 2.0 eq, 1.2 mmol) and piperidine (25.50 mg, 0.5 eq, 0.3 mmol) was stirred at 100° C. for overnight. Ater cooling to room temperature, the solid (350 mg) was filtered and washed with MeOH and dried. LCMS [416.4 (M+1)].

### Example 85

### 4-(4-Benzoylamino-phenylamino)-7-oxo-7,8-dihydro-pyrido[2,3-d]pyrimidine-6-carboxylic acid

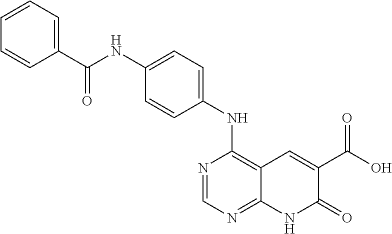

To a suspension of 4-(4-benzoylamino-phenylamino)-7-oxo-7,8-dihydro-pyrido[2,3-d]pyrimidine-6-carboxylic acid methyl ester (00 mg, 0.24 mmol) in THF/MeOH (2 mL/2 mL) was added aq. NaOH (1N, 0.48 mL, 2 eq., 0.48 mmol) was stirred 3 h at 50° C. Removal of the solvents, the mixture was neutralized with aqueous HCl (1N, 1 mL). The precipitate (85 mg) was filtered and dried. LCMS [402.3 (M+1)].

### Example 86

### 4-(4-Benzoylamino-phenylamino)-7-oxo-7,8-dihydro-pyrido[2,3-d]pyrimidine-6-carboxylic acid 2-(dimethylamino-ethyl)-amide

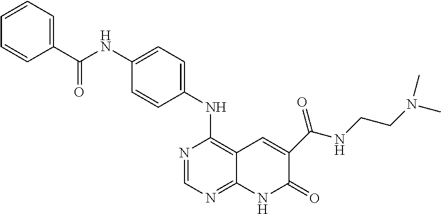

The mixture of 4-{[4-(benzoylamino)phenyl]amino}-7-oxo-7,8-dihydropyrido[2,3-d]pyrimidine-6-carboxylic acid (50.00 mg; 0.12 mmol; 1.00 eq.). EDCI (26.17 mg, 0.14 mmol, 1.1 eq), HOBt (18.52 mg, 0.14 mmol, 1.1 eq), N,N-dimethylethane-1,2-diamine (12.08 mg; 0.14 mmol; 1.10 eq.) and N-ethyl-N-isopropylpropan-2-amine (48.30 mg; 0.37 mmol; 3.00 eq.) in DMF (1.5 mL) was stirred for 24 h. The product (2 mg) was obtained through reverse phase HPLC. LCMS [472.4 (M+1)].

### Example 87

### 3,4-Difluoro-N-(4-(6-nitro-7-oxo-7,8-dihydro-1,8-naphthyridin-4-yl amino)phenyl)benzamide

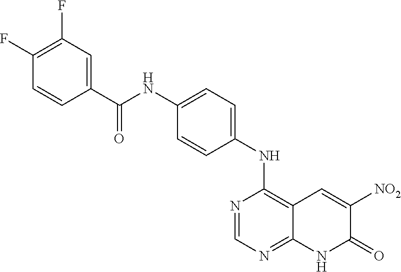

5-chloro-3-nitro-1,8-naphthyridin-2(1H)-one (600.00 mg; 2.66 mmol; 1.00 eq.) was suspended in ether (10 ml), was added 1N HCl ether (2 eq.). The reaction mixture was stirred at rt for 2 h. The solid was filtered and dried. The mixture of the salt, N-(4-aminophenyl)-4-fluoro-2-(trifluoromethyl)benzamide (726.24 mg; 2.93 mmol; 1.10 eq.), in NMP (3 mL) was stirred for overnight at 150 C for 2 h. After cooling to rt, the water was added and precitate was filtered, washed with water, MeOH, dried. 1.1 g of the desired product was obtained. LCMS [438.4 (M+1)].

### Example 88

### 4-Fluoro-2-trifluoromethyl-N-(4-(6-nitro-7-oxo-7,8-dihydro-1,8-naphthyridin-4-yl-amino)phenyl)benzamide

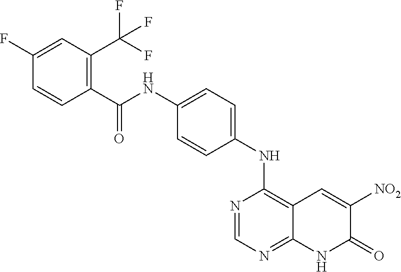

5-chloro-3-nitro-1,8-naphthyridin-2(1H)-one (200.00 mg; 0.89 mmol; 1.00 eq.) was suspended in ether (10 ml), was added 1N HCl ether (2 eq.). The reaction mixture was stirred at rt for 2 h. The solid was filtered and dried. The mixture of the salt, N-(4-aminophenyl)-4-fluoro-2-(trifluoromethyl)benzamide (290.85 mg; 0.98 mmol; 1.10 eq.), in NMP (3 mL) was stirred for overnight at 150 C for 2 h. After cooling to rt, the water was added and precitate was filtered, washed with water, MeOH, dried. 250 mg of the product was obtained. LCMS [488.3 (M+1)].

### Example 89

### 3,4-Difluoro-N-(4-(6-amino-7-oxo-7,8-dihydro-1,8-naphthyridin-4-yl-amino)phenyl)benzamide

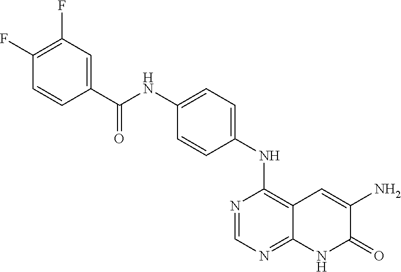

To a solution of 3,4-difluoro-N-{4-[(6-nitro-7-oxo-7,8-dihydro-1,8-naphthyridin-4-yl)amino]phenyl}benzamide (1.00 g) in DMF (minimium amount for dissolving SM)/MeOH (30 mL) was added Pd/C (100 mg) and the mixture was hydrogenated at 10 psi of H2 atmosphere for overnight. After filtering the solid through a pad of Celite and the solvent was removed under vacuo. Water (100 mL) was added and the precipitate was collected by filtration, dried. The product (600 mg) was obtained. LCMS [408.4 (M+1)].

### Example 90

### 4-Fluoro-2-trifluoromethyl-N-(4-(6-amino-7-oxo-7,8-dihydro-1,8-naphthyridin-4-yl-amino)phenyl)benzamide

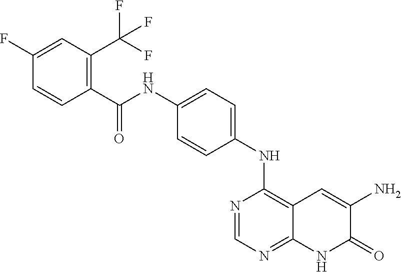

To a solution of 4-fluoro-3-2-trifluoromethyl-N-{4-[(6-nitro-7-oxo-7,8-dihydro-1,8-naphthyridin-4-yl)amino]phenyl}benzamide (50 mg) in DMF (minimium amount for dissolving SM)/MeOH (3 mL) was added Pd/C (20 mg) and the mixture was hydrogenated at 10 psi of H2 atmosphere for overnight. After filtering the solid through a pad of Celite and the solvent was removed under vacuo. Water (20 mL) was added and the precipitate was collected by filtration, dried. The product (40 mg) was obtained. LCMS [458.4 (M+1)].

### Example 91

### 4-Fluoro-N-[4-(5-hydroxy-7-oxo-7,8-dihydro-[1,8]naphthyridin-4-ylamino)-phenyl]-2 trifluoromethyl-benzamide

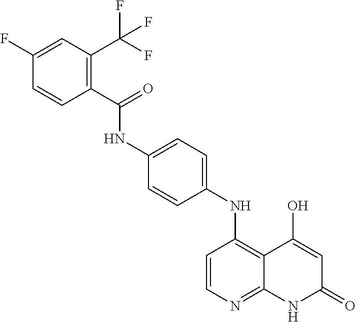

### Example 92

### N-[4-(5-Hydroxy-7-oxo-7,8-dihydro-[1,8]naphthyridin-4-ylamino)-phenyl]-benzamide

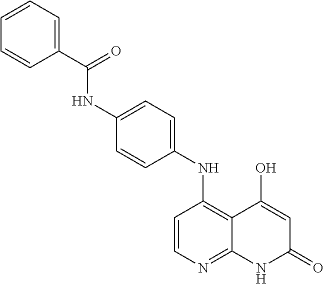

### Example 93

### N-[4-(5-Cyclopropylmethoxy-7-oxo-7,8-dihydro-[1,8]naphthyridin-4-ylamino)-phenyl]benzamide

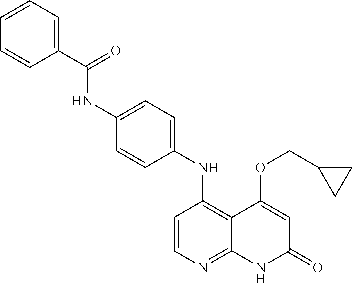

### Example 94

### N-[4-(5-Methyl-7-oxo-7,8-dihydro-[1,8]naphthyridin-4-ylamino)-phenyl]-benzamide

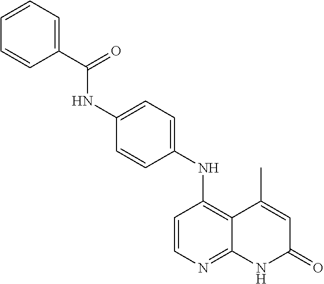

### Example 95

### 4-Fluoro-N-[4-(5-methyl-7-oxo-7,8-dihydro-[1,8]naphthyridin-4-ylamino)-phenyl]-2 trifluoromethyl-benzamide

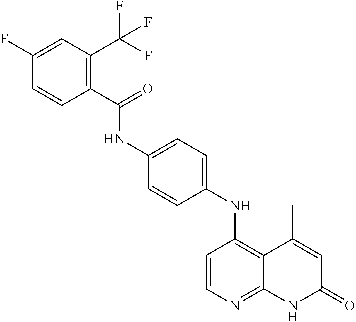

### Example 96

### 4-Fluoro-N-[4-(6-fluoro-7-oxo-7,8-dihydro-[1,8]naphthyridin-4-ylamino)-phenyl]-2 trifluoromethyl-benzamide

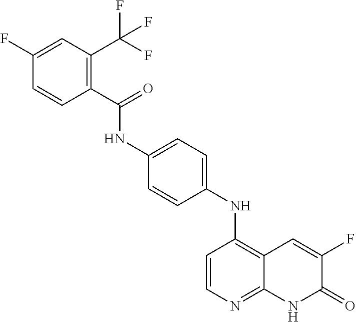

### Example 97

### Biochemical Enzyme Assays for Aurora Activity

Numerous models exist for identification of a signal transduction pathway and detection of interactions among various signal transduction pathways. For example, there are the cell culture models of Khwaja et al., EMBO, (1997), 16: 2783-93, and transgenic animal models of White et al., Oncogene, (2001), 20: 7064-7072. For the identification of certain stages in the signal transduction cascade, interacting compounds can be utilized in order to modulate the signal (see, for example, Stephens et al., Biochemical J., (2000), 351:95-105). The compounds according to the invention also can be used as reagents for testing kinase-dependent signal transduction pathways in animals and/or cell culture models, or in the clinical diseases mentioned herein.

Measurement of kinase activity is a technique well known to a person skilled in the art. Generic test systems for the determination of kinase activity that employ substrates (as for example, histone found in Alessi et al., FEBS Lett. (1996), 399(3): 333-338) or basic myelin protein are described in the literature (see for example, Campos-González, R. and Glenney, Jr., J. R., J. Biol. Chem. (1992), 267:14535).

For the identification of kinase inhibitors, various assay systems are available. In scintillation proximity assay (Sorg et al., J. of. Biomolecular Screening, (2002), 7:11-19) and flashplate assay, the radioactive phosphorylation of a protein or peptide as substrate with ATP is measured. In the presence of an inhibitory compound, a decreased radioactive signal, or none at all, is detectable. Homogeneous time-resolved fluorescence resonance energy transfer (HTR-FRET) and fluorescence polarisation (FP) technologies also are suitable as assay methods (Sills et al., J. of Biomolecular Screening, (2002) 191-214), as is the use of a caliper test known to those skilled in the art.

Other non-radioactive ELISA assay methods use specific phospho-antibodies (phospho-ABs). The phospho-AB binds only to the phosphorylated substrate. This binding then can be detected by chemiluminescence using a second peroxidase-conjugated anti-sheep antibody (Ross et al., Biochem. J. (2002)).

The Aurora assays described here were performed on two Caliper Life Sciences systems, the LC3000 and the Desktop Profiler. These provide data on enzyme activity via measurement of the relative amounts of phosphorylated or unphosphorylated fluorescently labelled substrate peptide at the end of an enzymatic reaction. These different states of peptide are resolved by applying a potential difference across the sample. The presence of a charged phosphate group on the product (as opposed to the substrate) causes a different peptide mobility between the two peptides. This is visualized by excitation of a fluorescent label on the substrate and product peptides and represented as peaks within the analysis software.

### LC3000 Method

In order to measure inhibitor activity of Aurora A inhibitors in the Caliper Life Sciences LC3000, a TTP Mosquito liquid handling instrument was used to place 0.25 μl of an appropriate concentration of inhibitor in 100% DMSO (for a dose response curve calculation) into each well of a 384-well plate. To this reaction components were added to a final volume of 25 μl:
0.067 ng/μl GST-Aurora A (Carna Biosciences 05-101. N-terminal GST fusion with full length Aurora A (1-403 amino acids), accession number NP_{—}940835.1).
15 μM ATP (Fluka, 02055)
1 mM DTT (Sigma, D0632)
1 mM MgCl2 (Sigma, M1028)
1 μM substrate peptide (sequence FITC-LRRASLG-((C═O)NH2), synthesized by Tufts Peptide Synthesis service.
100 mM HEPES pH 7.5 (Calbiochem, 391338)
0.015% Brij-35 (Sigma, B4184)

The reaction was incubated for 90 min at 25° C., and then stopped by the addition of 70 μl of Stop buffer (100 mM HEPES pH 7.5, 0.015% Brij-35, 10 mM EDTA (Sigma, E7889)).

The plate was read on a Caliper LC 3000 in an Off-Chip mobility shift assay format, using the following parameters for a 12-sipper chip: screening pressure −1.8 psi, upstream voltage −2700, downstream voltage −1000. These conditions cause unphosphorylated substrate and phosphorylated product peptide to resolve as separate peaks allowing direct measurement of percentage of conversion of substrate to product. The percent conversion was plotted against concentration of inhibitor to produce a sigmoidal dose response curve, from which an 1050 was calculated using XLFit for Microsoft Excel.

### Desktop Profiler Method

The Desktop Profiler utilizes the same prinicipal as the LC 3000 for calculating percentage conversion of a substrate to product. Caliper Life Sciences provided proprietary flash frozen pre-made 384 well plates containing selected kinases. Each column in the 384 well plate contained a particular selected kinase. A second plate, the ‘substrate plate’ contained a mix of fluorescently labeled peptide substrate and ATP. These were arranged in columns so that transfer for substrate plate to enzyme plate provided the correct enzyme with the correct substrate/ATP concentration.

Compounds were added to a thawed enzyme plate in the desired format, in single concentrations. Reactions were initiated by transfer of the substrate/ATP mix from the substrate plate. The enzyme plate was incubated for 90 mins at 25° C. The reaction was stopped by addition of 70 μl of Stop Buffer (100 mM HEPES pH 7.5, 0.015% Brij-35, 10 mM EDTA (Sigma, E7889)).

Plates were read in a manner identical to that of the LC3000, and the ratio between substrate and product peaks provided the activity of the enzyme in that well. This was best represented by a plate heat map which colors each well by percent inhibition as compared to positive and negative controls (no inhibitors and no ATP respectively).

The results of compounds tested on either of these two systems are given in Table 1 below:

TABLE 1

| | Aurora Kinase A** | Aurora Kinase B** |
|---|---|---|
| Compound* | IC₅₀ in nM | IC₅₀ in nM |
| 5 | + | + |
| 6 | + | + |
| 7 | + | |
| 8 | + | |
| 9 | + | + |
| 10 | ++ | |
| 11 | + | |
| 12 | + | |
| 13 | + | |
| 14 | + | |
| 15 | + | |
| 16 | + | |
| 17 | + | |
| 18 | + | |
| 19 | + | |
| 20 | + | |
| 21 | + | |
| 22 | +++ | |
| 23 | ++++ | |
| 24 | ++++ | |
| 25 | + | |
| 26 | + | |
| 27 | + | |
| 28 | + | |
| 29 | ++ | |
| 30 | + | |
| 31 | + | |
| 31a | + | |
| 32 | + | |
| 33 | + | |
| 34 | + | |
| 35 | + | |
| 36 | + | |
| 37 | +++ | +++ |
| 38 | ++++ | ++++ |
| 39 | ++++ | ++++ |
| 40 | + | |
| 41 | + | |
| 42 | + | |
| 43 | + | |
| 44 | ++ | |
| 45 | + | |
| 46 | + | |
| 47 | + | |
| 48 | ++++ | |
| 49 | ++++ | |
| 50 | ++++ | |
| 51 | ++++ | ++++ |
| 52 | + | |
| 53 | ++++ | |
| 54 | ++++ | |
| 55 | ++++ | |
| 56 | +++ | |
| 57 | ++++ | ++ |
| 58 | ++++ | +++ |
| 59 | ++++ | + |
| 60 | + | |
| 61 | ++++ | |
| 62 | ++++ | |
| 63 | + | |
| 64 | + | |
| 65 | ++++ | |
| 66 | + | |
| 67 | + | |
| 68 | + | |
| 69 | ++++ | |
| 70 | ++++ | |
| 71 | + | |
| 72 | + | |
| 73 | + | |
| 74 | + | |
| 75 | + | |
| 76 | + | |
| 77 | ++++ | |
| 78 | + | |
| 79 | ++++ | |
| 80 | + | |
| 81 | ++ | |
| 82 | + | |
| 83 | ++++ | |
| 84 | ++++ | |
| 85 | ++++ | |
| 86 | ++++ | +++ |
| 87 | + | |
| 88 | + | |
| 89 | + | + |
| 90 | + | + |
| 91 | + | + |
| 92 | + | + |
| 93 | + | + |
| 94 | + | + |
| 95 | + | + |
| 96 | + | + |

*Compound Nos. 1-4 purposely omitted.
**IC₅₀ ranges are given as: + = 1-300 nM ++ = 301-600 nM +++ = 601-1000 nM ++++ = >1001 nM

All publications and patent applications cited in this specification hereby are incorporated by reference. Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit and scope of the invention.

We claim:

1. A compound of the Formula III, IV, or I: 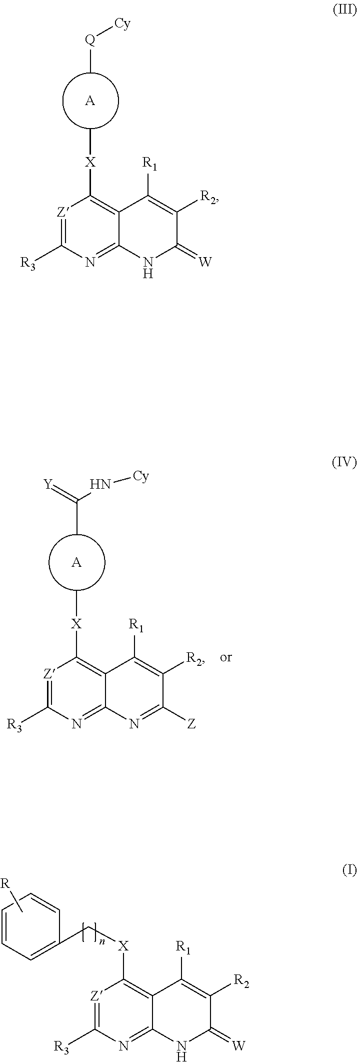
wherein:
X is NH, NH—C(═O), (—C═O)NH, NH—C(═O)NH, O, S, SO₂NH, CH₂, —C≡C—, or —HC═CH
Q is NH(C═Y) or (C═Y)NH;
Y and W each independently is O, S, or NH;
R is H, halo, cyano, nitro, alkyl, trifluoromethyl, heteroalkyl, OR′, SR′ and NR′R″, where R′ and R″ each independently are H, alkyl, haloalkyl, alkylhalo, or heteroalkyl; or R is an heteroalkyl chain that optionally is bound at either end to adjoining carbon atoms of the phenyl ring to which it is attached, thereby forming a bicyclic ring structure;
R₁, R₂, R₃ each independently is H, SH or an ether or oxidated form of sulfur; halo, nitro, amino, alkyl, formyl, hydroxy, hydroxyalkyl, alkoxy, cyano, carboxy, carboxylic acid, a carboxylic acid ester, a carboxylic acid amide such as —(C═O)—(NrxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid; wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group;
n is 1, 2, 3 or 4, with the proviso that n may=0 when X is other than oxygen;
A is a 3-7 membered ring, saturated or unsaturated, optionally having 1 or more heteroatoms and further optionally substituted;
Cy is selected from the group consisting of an unsubstituted or substituted cycloalkyl, bicycloalkyl, aryl, heterocycle and heteroaryl;
Z is H, SH, hydroxy, halo, amino, acyl, formyl, alkylamino-heterocycle, dialkylamino-heterocycle, alkylamino-alkylamino, dialkylamino-alkylamino, alkylamino-alkoxy, dialkylamino-alkoxy, heterocyclic alkoxy, C₁₋₆ alkyl ester, phenyl, benzoyl, phenyl alkyl ketone, alkyl propanoyl, dialkyl alkanamide, acetic acid, or acetic acid amides;
Z′ is CH or N; or
a pharmaceutically acceptable salt, prodrug, hydrate, solvate, racemic mix, tautomer or enantiomer thereof.

2. The compound of claim 1, Formula (I), wherein:
X is NH, NH—C(═O), (—C═O)NH, NH—C(═O)NH, O, S, SO₂NH, CH₂, —C≡C—, or —HC═CH;
W is O, S, CH₂, or NH;
R is H, halo, cyano, nitro, alkyl, trifluoromethyl, heteroalkyl, OR′, SR′ and NR′R″, where R′ and R″ each independently are H, alkyl, haloalkyl, alkylhalo, or heteroalkyl; or R is an heteroalkyl chain that optionally is bound at either end to adjoining carbon atoms of the phenyl ring to which it is attached, thereby forming a bicyclic ring structure;
R₁, R₂, R₃, each independently is H, SH or an ether or oxidated form of sulfur; halo, nitro, amino, alkyl, formyl, hydroxy, hydroxyalkyl, alkoxy, cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid, wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group;
n is 1, 2, 3 or 4, with the proviso that n may=0 when X is other than oxygen; or
a pharmaceutically acceptable salt, prodrug, hydrate, solvate, racemic mix, tautomer or enantiomer thereof.

3. The compound of claim 2 wherein the residues not designated in greater detail have the meaning indicated in claim 2, but in which
in Subformula Ia W is O, X is NH, n=0, and R is H;
in Subformula Ib W is O, X is NH, n=0, and R simultaneously is Cl and a 1,3 dioxoalkylene chain bound to the phenyl ring so as to form 1,3-dioxolane;
in Subformula Ic W is O, X is O, n=1, and R is H;
in Subformula Id W is S, X is CH₂, n=0, and R is Cl;
in Subformula Ie W is NH, X is CH₂, n=0, and R is F;
in Subformula If W is O, X is NH, n=1, and R is di-fluoro.

4. The compound of claim 1, Formula III, wherein:
X is NH, NH—C(═O), NH—CH₂, (—C═O)NH, NH—C(═O)NH, O, S, SO₂NH, CH₂, —C≡C—, or —HC═CH—;
Q is NH(C═Y) or (C═Y)NH;
Y and W each independently is O, S, or NH;
A is a 3-7 membered ring, saturated or unsaturated, optionally having 1 or more heteroatoms and further optionally substituted;
Cy is selected from the group consisting of an unsubstituted or substituted cycloalkyl, bicycloalkyl such as norbornyl, aryl, heterocycle and heteroaryl;
R₁, R₂, R₃, each independently is H, SH or an ether or oxidated form of sulfur; halo, nitro, amino, alkyl, formyl, hydroxy, hydroxyalkyl, alkoxy, cyano, carboxy,
carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid, wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or
a pharmaceutically acceptable salt, prodrug, hydrate, solvate, tautomer, racemic mix, or enantiomer thereof.

5. The compound of claim 4 wherein the residues not designated in greater detail have the meaning indicated in claim 4, but in which
in Subformula IIIa Q is NH(C═O), W is O, X is NH, and A and Cy are phenyl;
in Subformula IIIb Q is NH(C═O) and Cy is methoxyphenyl;
in Subformula IIIc Q is NH(C═O) and Cy is methylphenyl;
in Subformula IIId Q is NH(C═O) and Cy is fluoro, trifluoromethyl phenyl;
in Subformula IIIe Q is NH(C═O) and Cy is chlorophenyl or dichlorophenyl;
in Subformula IIIf Q is NH(C═O) and Cy is naphthyl;
in Subformula IIIg Q is NH(C═O) and Cy is norbornyl;
in Subformula IIIh Q is NH(C═O) and Cy is trifluoromethoxyphenyl;
in Subformula IIIj Q is (C═O)NH, W is O, X is NH, and A and Cy are phenyl;
in Subformula IIIk Q is (C═O)NH and Cy is methoxyphenyl;
in Subformula IIIm Q is (C═O)NH and Cy is methylphenyl;
in Subformula IIIn Q is (C═O)NH and Cy is fluoro, trifluoromethyl phenyl;
in Subformula IIIo Q is (C═O)NH and Cy is chlorophenyl or dichlorophenyl;
in Subformula IIIp Q is (C═O)NH and Cy is naphthyl;
in Subformula IIIq Q is (C═O)NH and Cy is norbornyl;
in Subformula IIIr Q is (C═O)NH and Cy is trifluoromethoxyphenyl.

6. The compound of claim 1, Formula IV, wherein:
X is NH, NH—C(═O), NH—CH₂, (—C═O)NH, NH—C(═O)NH, O, S, SO₂NH, CH₂, —C≡C—, or —HC═CH—;
Y is O, S, or NH;
A is a 3-7 membered ring, saturated or unsaturated, optionally having 1 or more heteroatoms and further optionally substituted;
Cy is selected from the group consisting of an unsubstituted or substituted cycloalkyl, bicycloalkyl such as norbornyl, aryl, heterocycle and heteroaryl;
Z is H, SH, hydroxy, halo, amino, acyl, formyl, alkylamino-heterocycle, dialkylamino-heterocycle, alkylamino-alkylamino, dialkylamino-alkylamino, alkylamino-alkoxy, dialkylamino-alkoxy, heterocyclic alkoxy, C₁₋₆ alkyl ester, phenyl, benzoyl, phenyl alkyl ketone, alkyl propanoyl, dialkyl alkanamide, acetic acid, or acetic acid amides;
Z′ is C or N;
------ denotes the presence or absence of a bond;
R₁, R₂, R₃, each independently is H, SH or an ether or oxidated form of sulfur; halo, nitro, amino, alkyl, formyl, hydroxy, hydroxyalkyl, alkoxy cyano, carboxy, carboxylic acid, a carboxylic acid ester, a carboxylic acid amide such as —(C═O)—N(RxRy), acetic acid, acetic acid ester, acetic acid amide including an acetic acid amide having substitutions —(C═O)—N(RxRy), substituted or unsubstituted aryl, heterocyclic-alkoxy, substituted or unsubstituted heterocycle or heteroaryl, alkylamino, dialkylamino, alkylaminoalkyl, dialkylaminoalkyl, alkyldiamino, alkylaminoalkoxy, alkyldiaminoalkoxy, heterocyclic-alkoxy, alkylamino amide, dialkylamino amide carboxylic acid ester, hydroxyalkyl-hydroxy, hydroxy-alkylamide ester, dihydroxy-alkylamide ester, hydroxyalkylamide acetic acid; wherein Rx and Ry each independently may be H, alkyl, aminoalkyl, dialkylaminoalkyl, aryl-aminoalkyl, carbocycle-aminoalkyl, or heteroaryl-amino alkyl; and wherein the two groups bonded to the N atom of an aminoalkyl group may, together with the N atom to which they are bound, join to form a heterocyclic group; or
a pharmaceutically acceptable salt, prodrug, hydrate, solvate, tautomer, racemic mix, or enantiomer thereof.

7. The compound of claim 6 wherein the residues not designated in greater detail have the meaning indicated in claim 6, but in which
in Subformula IVa R₁ is H, X is NH, A is phenyl, Cy is phenyl, and Z is 4-dimethyl amino-piperidine;
in Subformula IVb R₁ is H, X is NH, A is phenyl, Cy is phenyl, and Z is dimethylamino-ethylamine;
in Subformula IVc Z is dimethylamino-propylamine;
in Subformula IVd Z is dimethylamino-pyrrolidine;
in Subformula IVe Z is dimethylaminoethoxy or dimethylaminopropoxy;
in Subformula IVf Z is pyrrolidinyl ethoxy or pyrrolidinyl propoxy;
in Subformula IVg W is O, Y is O, X is NH, A is phenyl, Cy is difluorophenyl, R₁ is phenyl and/or carboxylic acid;
in Subformula IVh W is O, Y is O, X is NH, A is phenyl, Cy is difluorophenyl, and R₁ is dimethylamino ethyl carboxylic acid amide;
in Subformula IVj W is O, Y is O, X is NH, A is phenyl, Cy is difluorophenyl, and Z is dihydroxypropyl carboxylic acid amide;
in Subformula IVk W is O, Y is O, X is NH, A is phenyl, Cy is difluorophenyl, and R₂ is methyl acetic acid;
in Subformula IVm W is O, Y is O, X is NH, A is phenyl, Cy is difluorophenyl, and R₂ is hydroxyethyl acetic acid amide.

8. A pharmaceutical composition comprising a compound of Formula III, IV or I according to claim 1, and a physiologically acceptable carrier, diluent, or excipient.

9. A composition of claim 8 wherein the compound is present in an amount of from about 0.1-1000 mg.

10. A composition of claim 9 wherein the compound is present in an amount from about 0.1-500 mg.

11. A composition of claim 8 that is the form of a tablet, capsule, powder, suspension, aerosol, spray, granulate, solution, or paste.

12. A composition of claim 8 that is administered orally, parenterally, intradermally, intranasally, subcutaneously, intrabuccally, intravenously, intramuscularly, or iontophoretically.

13. A process for preparing a compound of claim 1 comprising the steps of:
a. reacting a compound intermediate of the Formula V, VI, or VII: 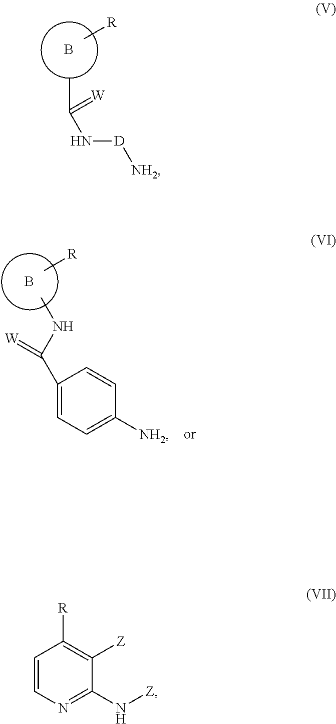
wherein:
B is 4-10 membered, saturated or unsaturated, ring that may be mono-, bi-, or tricyclic, and optionally may have one or more heteroatoms;
D is phenyl, a carbocycle, or a heterocycle, any of which optionally is substituted;
Z is H, SH, hydroxy, halo, amino, acyl, formyl, alkylamino-heterocycle, dialkylamino-heterocycle, alkylamino-alkylamino, dialkylamino-alkylamino, alkylamino-alkoxy, dialkylamino-alkoxy, heterocyclic alkoxy, C₁₋₆ alkyl ester, phenyl, benzoyl, phenyl alkyl ketone, alkyl propanoyl, dialkyl alkanamide, or acetic acid; and
R and W have the same meanings as given for Formulae I, II, III and IV;
with t-butyl COCl in the presence of TEA to provide a first intermediate product that is a t-butyl carboxamide substituted pyridine;
b. reacting the first intermediate product with butyl lithium in DMF to provide a second intermediate product that is an optionally substituted acetyl, t-butyl carboxamide substituted pyridine;
c. reacting the second intermediate product with t-butyl methyl ester in the presence of LDA to provide a third intermediate product that is a pyridine having t-butyl carboxylic acid ester hydroxymethyl and t-butyl carboxamide substituents, and that may be further substituted;
d. refluxing the third intermediate product with aqueous HCl to provide a fourth intermediate of the Formula VIII: 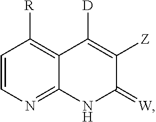
and
e. reacting the compound of Formula VIII with a bis-phenyl carboxamide wherein one phenyl is substituted by an amino group and the other phenyl is optionally substituted, in the presence of Pd and X-phosphate to provide the final product compound.

14. A method of treating a proliferative, autoimmune, anti-inflammatory or infectious disease disorder that comprises administering to a subject in need thereof a therapeutically effective amount of a compound of claim 1.

15. The method of claim 14 wherein the disorder is selected from the group consisting of angiogenesis, cancers, tumors, arteriosclerosis, ocular disease, arthritis, thrombosis, fibrosis, glomerulonephritis, psoriasis, restenosis, transplant rejection, cirrhosis, viral and bacterial infections, and autoimmune disease.

16. The method of claim 15 wherein the disease a cancer.

17. The method of claim 14 wherein the subject is a mammal.

18. The method of claim 17 wherein the mammal is a human.

19. The method of claim 14 wherein administration is simultaneous, sequential or in alternation with administration of at least one other active drug agent.

20. A kit comprising separate packets, the first having a therapeutically effective amount of a pharmaceutical composition according to claim 8, and the second having a therapeutically effective amount of a pharmaceutical composition comprising a further pharmaceutically active ingredient.

21. The compound of claim 1 selected from the group consisting of N-(4-aminophenyl)-2-fluoro-benzamide; N-(5-amino-pyrimidin-2-yl)-benzamide; N-(4-aminophenyl)-3-fluoro-benzamide; N-(4-aminophenyl)-2-trifluoromethyl-benzamide; N-(4-aminophenyl)-2-trifluoromethyl-benzamide; N-(4-aminophenyl)-4-trifluoromethyl-benzamide; N-(4-aminophenyl)-2-fluoro-3-trifluoromethyl-benzamide; N-(4-aminophenyl)-4-fluoro-2-trifluoromethyl-benzamide; N-(4-aminophenyl)-2,6-difluoro-benzamide; N-(4-aminophenyl)-3,4-difluoro-benzamide; N-(4-aminophenyl)-3,5-difluoro-benzamide; N-(4-aminophenyl)-2,4-difluoro-benzamide; cyclohexanecarboxylic acid (4-aminophenyl)-amide; N-(4-aminophenyl)-3,5-bis-trifluoromethyl-benzamide; naphthalene-2-carboxylic acid (4-aminophenyl)-amide; N-(4-aminophenyl)-2-methoxy-benzamide; N-(4-aminophenyl)-4-methyl-benzamide; N-(4-aminophenyl)-2-fluoro-4-trifluoromethyl-benzamide; N-(4-aminophenyl)-3-fluoro-5-trifluoromethyl-benzamide; N-(4-aminophenyl)-4-chloro-benzamide; N-(4-aminophenyl)-4-trifluoromethoxy-benzamide; N-(4-aminophenyl)-2-methyl-benzamide; N-(4-aminophenyl)-3-methyl-benzamide; naphthalene-1-carboxylic acid (4-aminophenyl)-amide; N-(4-aminophenyl)-2,6-dichloro-benzamide; N-(4-aminophenyl)-3,4-dichloro-benzamide; N-(4-aminophenyl)-2,4-dichloro-benzamide; N-(4-chloropyridin-2-yl)-2,2-dimethylpropanamide; N-(4-chloro-3-formylpyridin-2-yl)-2,2-dimethylpropanamide; tert-butyl 3-{4-chloro-2-[(2,2-dimethylpropanoyl)amino]pyridin-3-yl}-3-hydroxypropanoate; 5-chloro-1,8-naphthyridi-2(1H)-one; N-(4-(7-oxo-7,8-dihydro-1,8-naphthyridin-4-yl-amino)phenyl)benzamide; N-(4-(7-oxo-7,8-dihydro-1,8-naphthyridin-4-yl-amino)pyrimidin-2-yl)benzamide; 2-fluoro-N-(4-(7-oxo-7,8-dihydro-1,8-naphthyridin-4-yl-amino)phenyl)benzamide; 3-fluoro-N-(4-(7-oxo-7,8-dihydro-1,8-naphthyridin-4-yl-amino)phenyl)benzamide; 4-fluoro-N-(4-(7-oxo-7,8-dihydro-1,8-naphthyridin-4-yl-amino)phenyl)benzamide; 2-trifluoromethyl-N-(4-(7-oxo-7,8-dihydro-1,8-naphthyridin-4-yl-amino)phenyl)benzamide; 4-trifluoromethyl-N-(4-(7-oxo-7,8-dihydro-1,8-naphthyridin-4-yl-amino)phenyl)benzamide; 2-fluoro-3-Trifluoromethyl-N-(4-(7-oxo-7,8-dihydro-1,8-naphthyridin-4-yl-amino)phenyl)benzamide; 4-fluoro-2-trifluoromethyl-N-(4-(7-oxo-7,8-dihydro-1,8-naphthyridin-4-yl-amino)phenyl)benzamide; 2,6-difluoro-N-(4-(7-oxo-7,8-dihydro-1,8-naphthyridin-4-yl-amino)phenyl)benzamide; 3,4-difluoro-N-(4-(7-oxo-7,8-dihydro-1,8-naphthyridin-4-yl-amino)phenyl)benzamide; 3,5-difluoro-N-(4-(7-oxo-7,8-dihydro-1,8-naphthyridin-4-yl-amino)phenyl)benzamide; 2,4-difluoro-N-(4-(7-oxo-7,8-dihydro-1,8-naphthyridin-4-yl-amino)phenyl)benzamide; N-(4-(7-oxo-7,8-dihydro-1,8-naphthyridin-4-yl-amino)phenyl)cyclohexanecarboxamide; N-(4-(7-oxo-7,8-dihydro-1,8-naphthyridin-4-yloxy)phenyl)benzamide; N-(2-(7-oxo-7,8-dihydro-1,8-naphthyridin-4-ylamino)phenyl)benzamide; 4-(7-oxo-7,8-dihydro-1,8-naphthyridin-4-ylamino)-N-phenyl-benzamide; 3,5-bis(trifluoromethyl)-N-(4-(7-oxo-7,8-dihydro-1,8-naphthyridin-4-yl-amino)phenyl)benzamide; N-naphthalen-2-yl-4-(7-oxo-7,8-dihydro-1,8-naphthyridin-4-yl-amino)benzamide; 2-methoxy-N-(4-(7-oxo-7,8-dihydro-1,8-naphthyridin-4-yl-amino)phenyl)benzamide; 4-methyl-N-(4-(7-oxo-7,8-dihydro-1,8-naphthyridin-4-yl-amino)phenyl)benzamide; 2-fluoro-4-trifluoromethyl-N-(4-(7-oxo-7,8-dihydro-1,8-naphthyridin-4-yl-amino)phenyl)benzamide; 3-fluoro-5-trifluoromethyl-N-(4-(7-oxo-7,8-dihydro-1,8-naphthyridin-4-yl-amino)phenyl)benzamide; 4-chloro-N-(4-(7-oxo-7,8-dihydro-1,8-naphthyridin-4-yl-amino)phenyl)benzamide; 4-trifluoromethoxy-N-(4-(7-oxo-7,8-dihydro-1,8-naphthyridin-4-yl-amino)phenyl)benzamide; 2-methyl-N-(4-(7-oxo-7,8-dihydro-1,8-naphthyridin-4-yl-amino)phenyl)benzamide; 3-methyl-N-(4-(7-oxo-7,8-dihydro-1,8-naphthyridin-4-yl-amino)phenyl)benzamide; N-naphthalen-1-yl-4-(7-oxo-7,8-dihydro-1,8-naphthyridin-4-yl-amino)benzamide; 2,6-dichloro-N-(4-(7-oxo-7,8-dihydro-1,8-naphthyridin-4-yl-amino)phenyl)benzamide; 3,4-dichloro-N-(4-(7-oxo-7,8-dihydro-1,8-naphthyridin-4-yl-amino)phenyl)benzamide; 2,4-dichloro-N-(4-(7-oxo-7,8-dihydro-1,8-naphthyridin-4-yl-amino)phenyl)benzamide; N-(4-(7-chloro-1,8-naphthyridin-4-yl-amino)phenyl)benzamide; N-(4-(7(4-dimethylamino-piperidin-1-yl-1,8-naphthyridin-4-yl-amino)phenyl)benzamide; N-(4-(7-(4-dimethylamino-ethylamino)-1,8-naphthyridin-4-yl-amino)phenyl)benzamide; N-(4-(7-(4-dimethylamino-propylamino)-1,8-naphthyridin-4-yl-amino)phenyl)benzamide; N-(4-(7-(4-dimethylamino-pyrrolidin-1-yl)-1,8-naphthyridin-4-yl-amino)phenyl)benzamide; N-(4-(7-(4-dimethylamino-ethoxy)-1,8-naphthyridin-4-yl-amino)phenyl)benzamide; N-(4-(7-(4-dimethylamino-propoxy)-1,8-naphthyridin-4-yl-amino)phenyl)benzamide; N-(4-(7-(2-pyrrolidin-1-yl-ethoxy)-1,8-naphthyridin-4-yl-amino)phenyl)benzamide; N-(4-(7-(3-morpholin-4-yl-propoxy)-1,8-naphthyridin-4-yl-amino)phenyl)benzamide; N-(4-(7-(2-morpholin-4-yl-ethoxy)-1,8-naphthyridin-4-yl-amino)phenyl)benzamide; N-(3-benzoyl-4-chloropyridin-2-yl)-2,2-dimethylpropanamide; (2-amino-4-chloro-pyridin-3-yl)-phenyl-methanone; 5-chloro-2-oxo-4-phenyl-1,2-dihydro-1,8-naphthyridi-3-carboxylic acid tert-butyl ester; 5-[(3,4-difluoro-benzoylamino)-phenylamino]-2-oxo-4-phenyl1,2-dihydro-1,8-naphthyridine-3-carboxylic acid; 5-[(3,4-difluoro-benzoylamino)-phenylamino]-2-oxo-4-phenyl-1,2-dihydro-1,8-naphthyridine-3-carboxylic acid (2-dimethylamino-ethyl)-amide; 5-[(3,4-difluoro-benzoylamino)-phenylamino]-2-oxo-4-phenyl-1,2-dihydro-1,8-naphthyridine-3-carboxylic acid (2-hydroxy-ethyl)-amide; 5-[(3,4-difluoro-benzoylamino)-phenylamino]-2-oxo-4-phenyl-1,2-dihydro-1,8-naphthyridine-3-carboxylic acid ((S)2,3-dihydroxypropyl)-amide; (5-chloro-2-oxo-1,2-dihydro-1,8-naphthyridin-3-yl)-acetic acid; 5-[(3,4-difluoro-benzoylamino)-phenylamino]-2-oxo-4-phenyl-1,2-dihydro-1,8-naphthyridine-3-acetic acid; and 5-[(3,4-difluoro-benzoylamino)-phenylamino]-2-oxo-4-phenyl-1,2-dihydro-1,8-naphthyridine-3-acetic acid (2-hydroxy-ethyl)-amide, 3,4-Difluoro-N-(4-(6-nitro-7-oxo-7,8-dihydro-1,8-naphthyridin-4-yl amino)phenyl)benzamide, 4-Fluoro-2-trifluoromethyl-N-(4-(6-nitro-7-oxo-7,8-dihydro-1,8-naphthyridin-4-yl-amino)phenyl)benzamide, 3,4-Difluoro-N-(4-(6-amino-7-oxo-7,8-dihydro-1,8-naphthyridin-4-yl-amino)phenyl)benzamide, 4-Fluoro-2-trifluoromethyl-N-(4-(6-amino-7-oxo-7,8-dihydro-1,8-naphthyridin-4-yl-amino)phenyl)benzamide, 4-Fluoro-N-[4-(5-hydroxy-7-oxo-7,8-dihydro-[1,8]naphthyridin-4-ylamino)-phenyl]-2-trifluoromethyl-benzamid, N-[4-(5-Hydroxy-7-oxo-7,8-dihydro-[1,8]naphthyridin-4-ylamino)-phenyl]-benzamide, N-[4-(5-Cyclopropylmethoxy-7-oxo-7,8-dihydro-[1,8]naphthyridin-4-ylamino)-phenyl]benzamide, N-[4-(5-Methyl-7-oxo-7,8-dihydro-[1,8]naphthyridin-4-ylamino)-phenyl]-benzamide, 4-Fluoro-N-[4-(5-methyl-7-oxo-7,8-dihydro-[1,8]naphthyridin-4-ylamino)-phenyl]-2 trifluoromethyl-benzamide, 4-Fluoro-N-[4-(6-fluoro-7-oxo-7,8-dihydro-[1,8]naphthyridin-4-ylamino)-phenyl]-2 trifluoromethyl-benzamide.
